(19) United States
(12) Patent Application Publication
(10) Pub. No.: US 2014/0295417 A1
(43) Pub. Date: Oct. 2, 2014

(54) IDENTIFICATION OF ANTIBIOTIC RESISTANCE

(71) Applicants: Walter Freiherr von Stein, Duesseldorf (DE); Mirko Stange, Duesseldorf (DE)

(72) Inventors: Walter Freiherr von Stein, Duesseldorf (DE); Mirko Stange, Duesseldorf (DE)

(73) Assignee: MIACOM, Duesseldorf (DE)

(21) Appl. No.: 14/005,946

(22) PCT Filed: Mar. 19, 2012
(86) PCT No.: PCT/EP2012/054803

§ 371 Date: Dec. 9, 2013

(30) Foreign Application Priority Data
Mar. 18, 2011 (EP) 11158836.4

(51) Int. Cl. C12Q 1/68

(52) U.S. Cl. CPC C12Q 1/689
USPC 435/6.11

(57) ABSTRACT

The invention relates to a method and a kit for the detection of an antibiotic resistance in a predetermined micro-organism in a biological sample. The method according to the invention comprises the following steps: (a) contacting the biological sample with a first nucleic acid labelled with a first label and capable of selectively hybridizing with a nucleic acid in the micro-organism, (b) identifying the micro-organism by the detection of the presence of the first label in an individual cell of the micro-organism, (c) contacting the sample with at least one probe for detection of an antibiotic resistance in a micro-organism, wherein said at least one probe is labelled with at least a second label, and (d) determining the antibiotic resistance of the micro-organism by the detection of the presence of at least the second label, wherein steps (b) and (d) are performed simultaneously so as to allow quick identification of a pathogen directly from a sample without culturing and without prior amplification.

The invention relates to a method and a kit for the detection of an antibiotic resistance in a predetermined micro-organism in a biological sample.

The characterisation of micro-organisms in routine diagnostic procedures encompasses the determination of a species' identity and its sensitivity towards antibiotics. In order to achieve this, micro-organisms need to be taken from their environment and enriched in a selective environment for the separate identification (ID) and antibiotic sensitivity testing (AST). Currently the AST/ID of micro-organisms is achieved by identifying presence or absence of an array of biochemical features and the (non-) capability to grow in the presence of antibiotics. Alternatively DNA can be extracted from a sample and the then pooled DNA is tested for the presence/absence of specific sequences utilising gene amplification techniques. This can signal the presence of an organism in the sample. Equally, the presence of a gene coding for antibiotic resistance in the sample can be detected. By definition, extracting DNA directly from a sample renders pooled DNA from an unknown mixture of cells. Unequivocal results can only be achieved if the DNA is extracted from a pure colony.

Resistance of micro-organisms against antibiotics can be mediated by one of the following mechanisms:
1. Some antibiotics disturb cell wall synthesis in a micro-organism. Resistance against such antibiotics can be mediated by altering the target of the antibiotic, namely a cell wall protein. For example, MRSA or/and ORSA produce an atypical Penicillin Binding Protein (PBP2a), which has a reduced penicillin binding capacity, or does not bind penicillin.
2. The antibiotic is inactivated before reaching the target. An example of this resistance mechanism is the presence of enzymes capable of inactivating the antibiotic by cleavage. For example, beta-lactamase is capable of cleaving the beta-lactam of penicillin or/and carbapenems. Another example is inactivation by binding to a protein so that the antibiotic can no more reach its target.
3. The antibiotic is removed from the cell before reaching the target by a specific pump. An example is the RND transporter.
4. Some antibiotics act by binding to ribosomal RNA (rRNA) and interact with protein biosynthesis in the micro-organism. A micro-organism resistant against such antibiotic may comprise a mutated rRNA having a reduced binding capability to the antibiotic, but having an essentially normal function within the ribosome.

Staphylococcus aureus is one of the most common causes of nosocomial or community-based infections, leading to serious illnesses with high rates of morbidity and mortality. In recent years, the increase in the number of bacterial strains that show resistance to methicillin or/and Oxacillin, methicillin resistant Staphylococcus aureus (MRSA) and oxacillin resistant Staphylococcus aureus (ORSA) have become a serious clinical and epidemiological problem because these antibiotics (or analogues) are considered the first option in the treatment of staphylococci infections. The resistance to these antibiotics implies resistance to many β-lactam antibiotics, in particular with low affinity for penicillins. For these reasons, accuracy and promptness in the detection of methicillin resistance or/and oxacillin resistance is of key importance to ensure correct antibiotic treatment in infected patients as well as control of MRSA or/and ORSA isolates in hospital environments, to avoid them spreading.

Methicillin-resistant Staphylococcus aureus are also termed multiple-resistant Staphylococcus aureus (MRSA), multidrug-resistant Staphylococcus aureus (MRSA). Methicillin resistance largely overlaps with oxacillin resistance. In other words, a MRSA may be an oxacillin-resistant Staphylococcus aureus (ORSA), and vice versa.

MRSA or/and ORSA strains harbour the mecA gene, which encodes a modified PBP2 protein (termed PBP2′ or PBP2a) with low affinity for methicillin and many β-lactam antibiotics, in particular with low affinity for penicillins. Phenotypic expression of methicillin resistance may depend on the growth conditions for S. aureus, such as temperature or osmolarity of the medium, and this may affect the accuracy of the methods used to detect methicillin resistance (1). Hetero-resistant bacterial strains may evolve into fully resistant strains and therefore be selected in those patients receiving 11-lactam antibiotics, thus causing therapeutic failure. From a clinical point of view they should, therefore, be considered fully resistant.

There are several methods for detecting methicillin resistance (1,9) including classical methods for determining a minimum inhibitory concentration MIC (disc diffusion, Etest, or broth dilution), screening techniques with solid culture medium containing oxacillin, and methods that detect the mecA gene or its protein product (PBP2′ protein) (3,4). Detection of the mecA gene is considered as the reference method for determining resistance to methicillin (1). However, many laboratories throughout the world do not have the funds required, the capacity or the experienced staff required to provide molecular assays for detecting MRSA or/and ORSA isolates. It is therefore essential that other, more useful, screening methods are incorporated into routine clinical practice. Moreover, the presence of antibiotic resistance has it's relevance at several levels, all of which are of clinical significance:
1. Presence of a gene conveying resistance, such as mecA, mef(E);
2. Presence of a repressor gene inhibiting the phenotypic expression of said resistance mechanism; e.g. MecA repressor;
3. Multiple resistance mechanisms; e.g. Macrolide resistance via modification of the ribosomal binding site and presence of efflux mechanism(s); and
4. Level of expression of said resistance mechanism regulated via transcription and translation detectable as the phenotype.

Current cultural techniques require the isolation of a discrete colony and the subsequent identification and resistance testing, assuming that a single colony is derived from a single cell and is therefore deemed to be pure. In reality however, the generation of a pure colony from a clinical sample, where pathogens frequently live in bio-film communities, cannot be guaranteed. Equally, using amplification technologies, nucleic acid sequences from multiple cells are extracted and amplified and can therefore render false positive results. Only if identification and resistance can be performed and be read on individual cells, is it possible to a true picture of the invading pathogen.

The increasing spread of antibiotic resistance in both community and healthcare systems necessitates the precision and speed of molecular biology. However, the complexity and cost of these assays prohibits the widespread application in a routine testing environment.

Taking into account the difficulties in identifying a micro-organism and its potential resistance against an antibiotic in a biological sample, it is the objective of the invention to provide a method and a kit for enabling quick identification of a pathogen directly from a sample without culturing and without amplification and in addition for enabling detection or exclusion of the presence of resistance towards an antibiotic of choice.

The objective is achieved by a method for the detection of an antibiotic resistance in a predetermined micro-organism in a biological sample, comprising the steps:
(a) contacting the biological sample with a first nucleic acid labelled with a first label and capable of selectively hybridizing with a nucleic acid in the micro-organism under conditions wherein the nucleic acids selectively hybridize with each other,
(b) identifying the micro-organism by the detection of the presence of the first label in an individual cell of the micro-organism,
(c) contacting the sample with at least one probe or at least one substrate for detection of an antibiotic resistance in a micro-organism, wherein said at least one probe is labelled with a second label, a third label or a fourth label, and wherein said at least one substrate can be modified by a resistance factor, and
(d) determining the antibiotic resistance of the micro-organism by the detection of the presence of one or more of the following labels, the second label, the third label and the fourth label, or/and the modified substrate in an individual cell of the micro-organism,
   wherein steps (b) and (d) are performed simultaneously.

The method according to the invention enables quick identification of a pathogen directly from a sample without culturing and without amplification. Moreover, using this method the presence of resistance towards an antibiotic of choice can be easily detected or excluded. The method according to the invention further enables identification of the micro-organism and antibiotic resistance testing on a cellular level. This reduces the complexity of the assays so that an unambiguous assignment of a phenotype can be made for individual cells. The assays are designed to reduce handling and turnaround time to enable screening programmes such as the screening of all incoming patients for e.g. MRSA or/and ORSA.

The present invention exploits the fact that the resistance mechanisms against antibiotics are associated with the presence of specific proteins, specific forms of proteins (e.g. a mutated form of a wild-type protein, or modified proteins), or specific forms of nucleic acids (e.g. a mutated rRNA) within the cell. The present invention provides a method wherein a micro-organism is identified, and, at the same time, the antibiotic resistance with this micro-organisms is determined by determination of the presence of resistance factors, for example specific proteins, specific forms of proteins or/and specific forms of nucleic acids which are associated with the antibiotic resistance.

The underlying principle of the method according to the invention is that if an organism is sensitive or resistant to an antibiotic, it will markedly differ from its resistant or sensitive counterpart. The combination of the identification of the micro-organism and the detection of the antibiotic resistance within one assay, as provided by the present invention, improves the characterisation of micro-organisms in routine diagnostic procedures by increased speed and decreased costs. Furthermore, by fast detection of the antibiotic resistance profile of micro-organisms in a clinical sample, a specific therapy can be initiated at an early stage of infection. Fast identification and characterisation of antibiotic resistance could lead to early isolation of patients, thus leading to a reduction of antibiotic resistances in nosocomial infections. Furthermore, the fast detection of antibiotic resistances in a patient's sample can lead to selection of a specific antibiotic suspected to be active in this patient, resulting in a reduced use of expensive broad-spectrum antibiotics.

Also, patients suspected of being infected with an antibiotic-resistant micro-organism which could be harmful for other patients (e.g., MRSA or/and ORSA), should be isolated. In some countries, for example in the Netherlands, all patients are isolated upon arrival at a clinic. As soon as they are checked for MRSA or/and ORSA or other problem-causing infections, this isolation can be terminated. Early identification would lead to a shortened isolation phase. The present invention provides a method for detection of an antibiotic resistance which could be performed immediately upon arrival in the clinic. As the results are available within a few minutes, no isolation of patients being negative for MRSA or/and ORSA or other harmful infections is required at all, thus reducing costs.

In alternative embodiments of the invention, steps (a) and (c) are performed simultaneously or separately.

In a preferred embodiment of the invention, steps (a), (b), (c) and (d) are performed simultaneously.

In another preferred embodiment of the invention, said at least one probe in step (c) is selected from
(i) an antibody or/and a fragment thereof being labelled with the second label and capable of selectively binding to a resistance factor, wherein the sample is contacted with the antibody or/and the fragment thereof under conditions wherein the antibody or/and the fragment thereof selectively binds to the resistance factor,
(ii) second nucleic acids labelled with the third label and capable of hybridizing with an RNA coding for a resistance factor, wherein the sample is contacted with the second nucleic acid under conditions wherein the second nucleic acid selectively hybridizes with the RNA, and
(iii) knottins, cystine-knot proteins or/and aptamers labelled with the fourth label and capable of selectively binding to a resistance factor, wherein the sample is contacted with the knottin, cystine-knot protein or/and aptamer under conditions wherein the knottin, cystine-knot protein or/and aptamer selectively binds to the resistance factor.

In another preferred embodiment of the invention, said at least one substrate in step (c) can be modified by beta-lactamase, wherein the substrate is nitrocefin, and wherein the sample is contacted with the substrate under conditions wherein the resistance factor modifies the substrate.

In another preferred embodiment of the invention, the antibiotic resistance in the micro-organism is induced.

In another preferred embodiment of the invention, the antibody includes a primary antibody capable of selectively binding to the resistance factor, and a secondary antibody labelled with the second label, wherein the secondary antibody is capable of selectively binding to the primary antibody.

In another preferred embodiment of the invention, the antibody or/and the fragment thereof and the second label, or the knottin, cystine-knot protein or/and aptamer and the fourth label, are coupled to a bead.

In another preferred embodiment of the invention, an aggregate is formed in step (c), said aggregate comprising more than one micro-organism cell and at least one bead.

In another preferred embodiment of the invention, a plurality of beads is coupled to the micro-organism cell in step (c).

In another preferred embodiment of the invention, the antibiotic is selected from the group consisting of aminoglycosides, carbacephems, carbapenems, cephalosporins, glycopeptides, macrolides, monobactams, penicillines, beta-lactam antibiotics, quinolones, bacitracin, sulfonamides, tetracyclines, streptogramines, chloramphenicol, clindamycin, and lincosamide.

In another preferred embodiment of the invention, the resistance against a beta-lactam antibiotic is detected by an antibody or/and a fragment thereof specifically binding to beta-lactamase or/and PBP2a binding protein, by a nucleic acid specifically hybridizing with an RNA encoding for beta-lactamase or/and PBP2a binding protein, or by a knottin, a cystine knot protein or/and an aptamer specifically binding to beta-lactamase or/and PBP2a binding protein.

In another preferred embodiment of the invention, the micro-organism is a Methicillin Resistant Staphylococcus aureus (MRSA) or/and an Oxacillin Resistant Staphylococcus aureus (ORSA), and wherein the antibiotic resistance is detected by the expression of an altered Penicillin Binding Protein 2 or mecA protein.

The micro-organism is selected from Vancomycin Resistant Staphylococcus aureus (VRSA), Vancomycin Resistant Staphylococcus (VRS), Vancomycin Resistant Enterococci (VRE) and Vancomycin Resistant Clostridium difficile (VRCD), and wherein the antibiotic resistance is detected by the expression of a peptide selected from vanA protein, vanB protein, vanC protein or/and modified peptidoglycans comprising a D-alanine-D-lactate C-terminus.

The objective is further achieved by providing a kit suitable for detecting an antibiotic resistance in a predetermined micro-organism, comprising
(a) a first nucleic acid capable of selectively hybridizing with a nucleic acid in the micro-organism, wherein the first nucleic acid is labelled with a first label, and
(b) at least one probe or substrate for detection of an antibiotic resistance, said probe or substrate being selected from
   (i) an antibody or/and a fragment thereof, wherein the antibody or/and the fragment thereof is/are labelled with a second label and capable of selectively binding to a resistance factor,
   (ii) second nucleic acids labelled with a third label and capable of hybridizing with an RNA coding for a resistance factor,
   (iii) knottins, cystine-knot proteins or/and aptamers labelled with a fourth label and capable of selectively binding to a resistance factor, and
   (iv) a substrate which can be modified by beta-lactamase, wherein the substrate is nitrocefin.

The kit of the present invention is in particular suitable to be used in the method of the present invention. The components (a) or/and (b) of the kit may be components as described herein in the context of the method of the present invention. As indicated herein, the kit may comprise further components, such as a data sheet providing information about the amount of detectable label in at least one combination of micro-organism, antibiotic and label, or a sample of a predetermined micro-organism in its non-resistant or/and resistant form, e.g. for control purposes. The kit is in particular suitable for use in the detection of an antibiotic resistance in a predetermined micro-organism in particular in a biological sample.

In the present invention “antibiotic resistance” refers to a resistance of a micro-organism against an antibiotic when the antibiotic is administered to a subject in need thereof in a dose that is sufficient to successfully eliminate the micro-organism in its non-resistant form.

In the method according to the invention, step (a) refers to contacting the biological sample with a first nucleic acid capable of selectively hybridizing with a nucleic acid in the micro-organism, under conditions wherein the nucleic acid selectively hybridizes with the nucleic acid, wherein the first nucleic acid is labelled with a first label.

The biological sample may be any sample of biological origin, such as a clinical sample or food sample, suspected of comprising an antibiotic-resistant micro-organism.

The labelled nucleic acid may in particular be a labelled oligonucleotide, capable of specifically hybridising with a nucleic acid in the micro-organism under in-situ conditions. The labelled oligonucleotide may have a length of up to 50 nucleotides, for example from 10 to 50 nucleotides. The skilled person knows suitable labels. The labelled oligonucleotide may be a linear oligonucleotide. The skilled person knows suitable conditions wherein the nucleic acid selectively hybridizes with the nucleic acid. The labelled oligonucleotide may be a molecular beacon, as for example described in WO 2008/043543, the disclosure of which is included herein by reference. Suitable conditions for hybridisation of molecular beacons are for example described in WO 2008/043543, the disclosure of which is included herein by reference.

The first nucleic acid may comprise at least one sequence selected from RNA, DNA and PNA sequences. The first nucleic acid may further comprise at least one nucleotide analogue, such as a PNA nucleotide analogue. The first nucleic acid may comprise at least one ribonucleotide and at least one deoxyribonucleotide.

The nucleic acid which has not hybridized with the target sequence may be removed by washing. If the nucleic acid is a molecular beacon, removal can be omitted.

A molecular beacon, as used herein, can be a nucleic acid capable of forming a hybrid with a target nucleic acid sequence and capable of forming a stem-loop structure if no hybrid is formed with the target sequence, said nucleic acid comprising:
1. a nucleic acid portion comprising (a1)) a sequence complementary to the target nucleic acid sequence, and (a2) a pair of two complementary sequences capable of forming a stem and flanking the sequence (a1), and
2. an effector and an inhibitor, wherein the inhibitor inhibits the effector when the nucleic acid forms a stem-loop structure, and wherein the effector is active when the nucleic acid is not forming a stem-loop structure.

The molecular beacon is also termed herein as “beacon”, “hairpin”, or “hairpin loop”, wherein the “open” form (no stem is formed) as well as the “closed” form (the beacon forms a stem) is included. The open form includes a beacon not forming a hybrid with a target sequence and a beacon forming a hybrid with the target sequence. Details of molecular beacons are disclosed in WO 2008/043543, the disclosure of which is included herein by reference.

The molecular beacon may have a length of at least 10, at least 15, at least 16, at least 17, at least 18, at least 19, or a least 20 nucleotides. The molecular beacon may have a length of at the maximum 30, at the maximum 40, or at the maximum 50 nucleotides.

It is preferred that hybridisation is performed at a temperature of between about 25° C. and about 65° C., in a more preferred embodiment the temperature is between about 35° C. and about 59° C. In an even more preferred embodiment, the temperature is at about 52° C. The incubation time is preferably between about 1 and about 30 minutes. It is more preferred to incubate for up to 15 minutes or for up to 10 minutes, or for about 15 minutes, or for about 10 minutes. After the incubation the carrier may be submerged in 50% ethanol followed by a bath in pure ethanol. Both steps may be run for between about 1 and about 10 minutes. The preferred length of incubation is between about 2 and about 6 minutes. It is more preferred to incubate about 4 minutes. The carrier may then be air-dried (e.g. on a hot plate) and the cells may be embedded in a balanced salt mounting medium.

The biological sample may be fixated or/and perforated before or/and during step (a). Fixation or/and perforation may be performed as described herein. The skilled person knows suitable protocols. Examples of fixation or/and perforation are described, for example in WO 2008/043543, the disclosure of which is included herein by reference.

Step (b) of the method of the present invention refers to identifying the micro-organism by the detection of the presence of the first label in the micro-organism.

“Identification” in the context of the present invention refers to identification of individual microbial cells as belonging to a particular taxonomic category, such as species, genus, family, class or/and order, etc. Identification can be performed based on morphological or/and biochemical classifications. The micro-organism may be selected from the group consisting of bacteria, yeasts, molds and eukaryotic parasites, in particular from Gram positive and Gram negative bacteria. Preferably, the predetermined micro-organism is selected from the group consisting of Staphylococcus, Enterococcus, Streptococcus and Clostridium. The predetermined Gram negative micro-organism may be selected from Enterobacteriaceae. The predetermined Gram negative micro-organism from the group of Enterobacteriaceae may be selected from Escherichia coli, Klebsiella spp., Proteus spp., Salmonella spp., and Serratia marcescens. The predetermined Gram negative micro-organism may also be selected from Pseudomonas aeruginosa, Acinetobacter spp., Burkholderia spp., Stenotrophomonas and Haemophilus influenzae.

More preferably, the predetermined micro-organism is selected from the group consisting of Methicillin resistant Staphylococcus, Oxacillin resistant Staphylococcus, Vancomycin resistant Staphylococcus, Vancomycin resistant Enterococcus, Vancomycin resistant Clostridium and high level Aminoglycoside resistant Enterococci.

The micro-organism is even more preferably selected from the group consisting of Staphylococcus aureus, Methicillin Resistant Staphylococcus aureus (MRSA), Oxacillin Resistant Staphylococcus aureus (ORSA), Vancomycin Resistant Staphylococcus aureus (VRSA), Vancomycin Resistant Staphylococcus (VRS), Vancomycin Resistant Enterococci (VRE), Streptococcus pneumoniae, drug resistant Streptococcus pneumoniae (DRSP), and Aminoglycoside resistant Enterococci (HLAR), Vancomycin resistant Clostridium difficile (VRCD).

Preferred is identification of the micro-organism by fluorescence in-situ hybridisation (FISH). An in-situ hybridisation protocol may be applied as laid down in patent application WO 2008/043543, which is incorporated herein by reference.

The first label may be detected by any suitable method known in the art. In particular, the first label may be detected by a detection method as described herein.

In the present invention, step (c) refers to contacting the sample with at least one probe for detection of an antibiotic resistance in a micro-organism. In the present invention, a “resistance factor” is a cellular component capable of mediating the resistance towards an antibiotic. Such resistance factor may be protein which is modified or altered in a micro-organism resistant against the antibiotic, compared with the protein in a non-resistant micro-organism. Examples of proteins being resistance factors include modified or altered target proteins of the antibiotic mediating the antibiotic action, for example a modified PBP2 protein (termed PBP2′ or PBP2a) with low affinity for methicillin and many fl-lactam antibiotics, in particular with low affinity for penicillins. Another example relates to vancomycin-resistant Staphylococcus aureus. In non-resistant Staphylococci, vancomycin binds to peptidoglycan precursors and thereby prevents cross-linking of the cell wall peptidoglycan. In Vancomycin-resistant Staphylococci, a D-alanyl-D-lactate ligase (VanA) modifies the D-Ala-D-Ala terminus to D-alanine-D-lactate (D-Ala-D-Lac). Vancomycin-resistant Staphylococci have a reduced capability of binding vancomycin to modified peptidoglycans comprising a D-alanine-D-lactate C-terminus so that vancomycin becomes ineffective.

Other examples of resistance factors refer to proteins capable of binding an antibiotic, thereby inactivating the antibiotic. Such antibiotic-binding proteins can be different from the target of the antibiotic. Binding of the antibiotic to a protein different from the target can result in resistance, as the antibiotic is no more capable of reaching the target.

Other examples of proteins being resistance factors include proteins capable of inactivating the antibiotic by its enzymatic activity (i.e. enzymes), for example a beta-lactamase.

Other examples of proteins being resistance factors include pumps capable of removing an antibiotic from a micro-organism cell, for example RND transporters.

In the present invention, the term “beta-lactamase” includes carbapenemase and NDM1.

In the present invention, the modification of a resistance factor being a protein may be a mutation, for example a deletion, insertion or amino acid exchange, compared with the unmodified protein present in a form which does not mediate antibiotic resistance. Also included are frameshift mutations.

The method of the present invention may comprise the induction of the antibiotic resistance in the micro-organism. An antibiotic resistance can be induced by contacting the micro-organism with a low concentration of an antibiotic, such as cephatoxin or cefoxitin. The antibiotic may be included in a clinical sample buffer, i.e. a buffer for keeping the micro-organisms obtained from a clinical sample. The clinical sample buffer can be prepared so that essentially no cell growth or/and cell division takes place. Contacting with the antibiotic may be performed in step (a), in step (b), in step (c), in step (d) or/and in an additional step of the method of the present invention. Contacting with the antibiotic may be performed in step (a), in step (b) in step (c) or/and in an additional step of the method of the present invention. Contacting with the antibiotic may also be performed in step (a), in step (b) or/and in an additional step of the method of the present invention. The additional step may be introduced between two of the steps of the method of the present invention, for example between steps (a) and (b), or/and between steps (b) and (c), or may be performed before the step (a).

In the present invention, “induction of antibiotic resistance” preferably indicates the expression of a resistance factor, as defined herein, against an antibiotic in a micro-organism capable of being resistant against this antibiotic. In the method of the present invention, induction of an antibiotic resistance may include induction of the expression of the resistance factor. “Induction of an antibiotic resistance”, as used herein, does preferably not include conditions allowing growth or/and propagation of the micro-organism. “Induction of an antibiotic resistance”, as used herein, does preferably not include conditions under which a non-resistant micro-organism acquires the genetic modification causing antibiotic resistance.

In particular, PBP2a can be induced, for example by cephatoxin or/and cefoxitin. Preferably, an antibiotic resistance in MRSA or/and ORSA is induced by induction of PBP2a.

The low concentration of the antibiotic employed in the method of the present invention is preferably below the concentration that is sufficient to successfully eliminate the micro-organism in its non-resistant form.

Examples of targets of antibiotics include rRNA. As described herein, many antibiotics act via binding to rRNA, thereby disturbing protein biosynthsis. In the present invention, the resistance factor may be a modified rRNA having a lower affinity for the antibiotic compared with the affinity of the unmodified rRNA in a non-resistant cell.

In the present invention, modification of a resistance factor being an rRNA may be a mutation, such as insertion, deletion, or/and nucleotide exchange. The mutation may be a point mutation or single nucleotide mutation. In particular, a modified rRNA being a resistance factor may comprise a point mutation. It is preferred to determine the resistance by point mutations in the 23S ribosomal RNA. It is also preferred to determine the resistance by point mutations in the 16S ribosomal RNA. The point mutations at different position in 23S or 16S rRNA induce resistance to a wide array of antibiotics such as macrolides, ketolides, tetracyclines, thiazolantibiotics, lincosamine, chloramphenicol, streptogram in, amecitin, animosycin, sparsoycin and puromycin. Detailed effects of respective point mutations are listed in Table 2. Point mutations at different positions of the 23S or 16S rRNA can generate an iso-phenotype. It would require an array of oligo-nucleotide probes to cover all possibilities. This invention offers a cost effective and efficient way of detecting antibiotic resistance mediated by rRNA irrespective of the position of the mutation.

In the present invention, the resistance factor may be selected from PBP2a, 3-lactamase, efflux transporters, wherein the efflux transporter is preferably selected from the group consisting of ATP-Binding Cassette (ABC) transporters, Major Facilitator Superfamily (MFS) transporters, Multidrug and Toxic Compound Extrusion (MATE) transporters and Resistance Nodulation Division (RND) transporters.

In the method of the present invention, the antibiotic resistance can be detected by mRNA encoding the resistance factor, if the resistance factor is a protein or polypeptide, as described herein. The mRNA may include a mutation, for example a deletion, insertion or amino acid exchange, compared with the mRNA encoding a polypeptide which is present in a form which does not mediate antibiotic resistance. Also included are frameshift mutations. In particular, the mRNA encodes PBP2a, p-lactamase, efflux transporters, wherein the efflux transporter is preferably selected from the group consisting of ATP-Binding Cassette (ABC) transporters, Major Facilitator Superfamily (MFS) transporters, Multidrug and Toxic Compound Extrusion (MATE) transporters and Resistance Nodulation Division (RND) transporters.

In the method of the present invention, a resistance against any antibiotic may be detected in a micro-organism. Preferably, the antibiotic is selected from the group consisting of aminoglycosides, carbacephems, carbapenems, cephalosporins, glycopeptides, macrolides, monobactams, penicillins, beta-lactam antibiotics, quinolones, bacitracin, sulfonamides, tetracyclines, streptogramines, chloramphenicol, clindamycin, and lincosamide.

More preferably, the antibiotics are selected from beta-lactam antibiotics, macrolides, lincosamide, and streptogramins. The antibiotic may also be selected from beta-lactam antibiotics. In particular the antibiotic may be selected from penicillins.

In the present invention, beta-lactam antibiotics in particular include carbapenems, cephalosporins, monobactams, and penicillines.

Even more preferably, the antibiotic is selected from the group consisting of Amikacin, Gentamicin, Kanamycin, Neomycin, Netilmicin, Streptomycin, Tobramycin, Loracarbef, Ertapenem, Imipenem, Cilastatin, Meropenem, Cefadroxil, Cefazolin, Cephalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefsulodine, Cefepime, Teicoplanin, Vancomycin, Azithromycin, Clarithromycin, Dirithromycin, Erythromycin, Roxithromycin, Troleandomycin, Aztreonam, Amoxicillin, Ampicillin, Azlocillin, Carbenicillin, Cloxacillin, Dicloxacillin, Flucloxacillin, Mezlocillin, Nafcillin, Penicillin, Piperacillin, Ticarcillin, Bacitracin, Colistin, Polymyxin B, Ciprofloxacin, Enoxacin, Gatifloxacin, Levofloxacin, Lomefloxacin, Moxifloxacin, Norfloxacin, Ofloxacin, Trovafloxacin, Mafenide, Prontosil, Sulfacetamide, Sulfamethizole, Sulfanilimide, Sulfasalazine, Sulfisoxazole, Trimethoprim, Trimethoprim sulfa, Sulfamethoxazole, Co-trimoxazole, Demeclocycline, Doxycycline, Minocycline, Oxytetracycline, Tetracycline, Chloramphenicol, Clindamycin, Ethambutol, Fosfomycin, Furazolidone, Isoniazid, Linezolid, Metronidazole, Mupirocin, Nitrofurantoin, Platensimycin, Pyrazinamide, Quinupristin/Dalfopristin, Rifampin, Spectinomycin, Amphotericin B, Flucanazole, Fluoropyrimidins, Gentamycin, Methicillin, Oxacillin and clavulanic acid.

Most preferably, the antibiotic is selected from Vancomycin, Methicillin, Oxacillin, Clindamycin, Trimethoprim, Trimethoprim sulfa, Gentamycin, and clavulanic acid.

Table 3 indicates resistance mechanisms against commonly known antibiotics in clinically relevant micro-organisms. Specific aspects of the present invention relate to the identification of a micro-organism and the detection of an antibiotic resistance of this micro-organism, wherein the combination of micro-organism and antibiotic resistance is selected from the combinations disclosed in Table 3.

In step (c)(i) an antibody or fragment thereof capable of selectively binding to a resistance factor may be contacted with the sample under conditions wherein the antibodies selectively binds to the resistance factor. Selective binding may be performed under in-situ conditions. The skilled person knows such conditions. Furthermore, the antibody is labelled with a second label. The skilled person knows suitable labels. The second label may be any label as described herein.

In the present invention, the antibody includes a primary antibody capable of selectively binding to the resistance factor, and a secondary antibody labelled with the second label, wherein the secondary antibody is capable of selectively binding to the primary antibody. The strategy of primary and secondary antibodies is well-known in the art. By this strategy, the signal detection can be improved.

In the present invention, the antibody, which is capable of selectively binding to a resistance factor may be generated using methods well known in the art. Such antibodies may include, but are not limited to, polyclonal, monoclonal, and chimeric single chain antibodies.

For the production of antibodies, various hosts including goats, rabbits, rats, mice, humans, and others, may be treated by injection with the resistance factor or any fragment thereof which has immunogenic properties. Depending on the host species, various adjuvants may be used to increase immunological response.

If the resistance factor is a polypeptide or protein, antibodies capable of specifically binding to the polypeptide or protein can be induced by a second polypeptide comprising a fragment of the polypeptide or protein having an amino acid sequence of at least five amino acids, and preferably at least 10 amino acids.

If the resistance factor is a nucleic acid, in particular an rRNA, antibodies capable of specifically binding to the nucleic acid can be induced by a second nucleic acid comprising a fragment of the nucleic acid having a sequence of at least five nucleotides, and preferably at least 10 nucleotides.

Monoclonal antibodies to the proteins may be prepared using any technique that provides for the production of antibody molecules by continuous cell lines in culture. These include, but are not limited to, the hybridoma technique, the human B-cell hybridoma technique, and the EBV-hybridoma technique (Köhler G. and Milstein C. (1975) Nature 256: 495-497; Kozbor D. et al. (1985) J. Immunol. Methods 81: 31-42; Cote R. J. et al., (1983) Proc. Natl. Acad. Sci. 80: 2026-2030; Cole S. P. et al., (1984) Mal Cell Biochem. 62: 109-120).

In addition, techniques developed for the production of ‘chimeric antibodies’, the splicing of mouse antibody genes to human antibody genes to obtain a molecule with appropriate antigen specificity and biological activity can be used (Morrison S. L. et al., (1984) Proc. Natl. Acad. Sci. 81: 6851-6855; Neuberger M. S. et al (1984) Nature 312: 604-608; Takeda S. et al., (1985) Nature 314: 452-454). Alternatively, techniques described for the production of single chain antibodies may be adapted, using methods known in the art, to produce single chain antibodies specific for the protein of the invention or a homologous protein. Antibodies with related specificity, but of distinct idiotypic composition, may be generated by chain shuffling from random combinatorial immunoglobulin libraries (Kang A. S. et al., (1991) Proc. Natl. Acad. Sci. 88: 11120-11123). Antibodies may also be produced by inducing in vivo production in the lymphocyte population or by screening recombinant immunoglobulin libraries or panels of highly specific binding reagents as disclosed in the literature (Orlandi R. et al., (1989) Proc. Natl. Acad. Sci. 86: 3833-3837; Winter G. and Milstein C., (1991) Nature 349: 293-299).

In the present invention, an antibody fragment, which is capable of selectively binding to a resistance factor may be generated using methods well known in the art. For example, such antibody fragments include, but are not limited to, the F(ab′)₂ fragments which can be produced by pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse W. D. et al., (1989) Science 246: 1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the protein and its specific antibody. A two-site, monoclonal-based immunoassay utilizing monoclonal antibodies reactive to two non-interfering protein epitopes are preferred, but a competitive binding assay may also be employed (Maddox D. E. et al., (1983) J. Exp. Med. 158: 1211-1216).

The labelled antibody not bound to the resistance factor may be removed by a wash step.

In one aspect, the antibody and the second label are coupled to a bead. The bead may have a diameter up to about 400 μm, preferably of about 10 nm to about 400 μm. The bead comprises a plurality of antibody molecules each capable of binding to the target. In step (c), aggregates comprising a bead and a micro-organism cell may be formed, wherein said micro-organism cell comprises a resistance factor to which the antibody selectively binds. By this strategy, removal of the antibodies not coupled to a resistance factor is facilitated compared with removal of free antibodies carrying a label.

In a further aspect, the bead comprises a plurality of second label molecules. Beads carrying a plurality of label molecules can be more readily detected, compared with an antibody molecule carrying a label.

In a particular aspect, the bead may be larger than the micro-organism cell. In this aspect, the bead may have a diameter of about 50 μm to about 400 μm, preferably about 100 μm to about 300 μm, more preferably about 150 μm to about 250 μm, most preferred about 200 μm. An aggregate may be formed in step (c), said aggregate comprising more than one micro-organism cell and at least one bead.

In a further particular aspect, the bead may be smaller than the micro-organism cell. In particular, the bead may be a micro-bead. The bead may have a diameter of up to 100 nm, preferably up to about 80 nm, more preferably up to about 60 nm, most preferred up to about 50 nm. Preferred bead have a diameter of about 20 nm or about 40 nm. In this aspect, in step (c), a plurality of beads may be coupled to a micro-organism cell.

In the method of the present invention, beads having antibodies coupled thereto, as described herein, may in particular be used for the detection of resistance factors located at the surface of the micro-organism cell.

In step (c)(ii), a second nucleic acid capable of hybridizing with an RNA coding for a resistance factor may be contacted with the sample under conditions wherein the nucleic acid selectively hybridizes with the RNA. The RNA coding for a resistance factor in particular is a mRNA. The skilled person knows suitable conditions wherein the nucleic acid selectively hybridizes with the RNA. For example, the labelled nucleic acid may in particular be a labelled oligonucleotide, capable of specifically hybridizing with a nucleic acid in the micro-organism under in-situ conditions. The labelled oligonucleotide may have a length of up to 50 nucleotides, for example from 10 to 50 nucleotides. The labelled oligonucleotide may be a linear oligonucleotide. The skilled person knows suitable conditions wherein the nucleic acid selectively hybridizes with the nucleic acid. The labelled oligonucleotide may be a molecular beacon, as disclosed herein. Suitable conditions for selective hybridisation of molecular beacons are for example described in WO 2008/043543, the disclosure of which is included herein by reference.

In situ hybridisation protocols, in particular FISH protocols, are described, for example, in Wilkinson, D. G. (ed.) “In situ Hybridisation. A practical approach”, second edition, “The practical approach series 196”, Oxford University Press, 1999, the disclosure of which are included herein by reference.

The second nucleic acid may comprise at least one sequence selected from RNA, DNA and PNA sequences.

The second nucleic acid may comprise at least one nucleotide analogue, such as a PNA nucleotide analogue.

The second nucleic acid may comprise at least one ribonucleotide and at least one deoxyribonucleotide.

The nucleic acid which has not hybridized with the target sequence may be removed by washing. If the nucleic acid is a molecular beacon, removal is not necessary.

In step (c)(iii), the sample may be contacted with the knottin, cystine-knot protein or/and aptamer under conditions wherein the knottin, cystine-knot protein or/and aptamer selectively binds to the resistance factor. The skilled person knows suitable conditions.

A knottin is a small disulfide-rich protein characterized by a “disulfide through disulfide knot”. The structure of knottins is for example described in http://knottin.cbs.cnrs.fr.

Cystine-knot protein are proteins providing a cystine-knot signature. The cystine-knot signature corresponds to the cystine-knot well described in the large family of transforming growth factors. The typical cysteine framework in these proteins consists of four cysteine residues with a cysteine spacing of Cys-(X)3-Cys and Cys-X-Cys, important for a ring structure formed by 8 amino acids. Two additional cysteines form a third disulfide bond which penetrates the ring structure, thus forming a cystine-knot. A description of cystine-knot proteins can be found on http://hormone.stanford.edu/cystine-knot.

Aptamers are oligonucleotide or peptide molecules which are designed for specifically binding to a target molecule. The skilled person knows suitable strategies for design or/and selection of aptamers.

The knottin, cystine-knot protein or/and aptamer and the fourth label may be coupled to a bead. The bead may be a bead, as described herein in the context of step (a).

In step (c)(iv), the sample may be contacted with substrates which can be modified by a resistance factor. The skilled person knows suitable substrates which can be modified by resistance factors. Step (c)(Iiv may be performed under in-situ conditions. For example, the substrate can be nitrocefin, which is modified by beta-lactamase. When cleaved by beta-lactamase, nitrocefin changes colour from yellow to red, which change can be detected in individual micro-organism cells by microscopy.

Step (d) of the method of the present invention refers to determination of the antibiotic resistance of the micro-organism by the detection of the presence of the second label, the third label, the fourth label, or/and the modified substrate in the micro-organism. The second, the third or/and the fourth label may be detected by any suitable method known in the art. In particular, the second, the third or/and the fourth label may be detected by a detection method as described herein, for example by a method described for detection of the first label.

The skilled person knows methods for identification of modified substrates. For example, nitrocefin cleaved by a beta-lactamase can be detected by a colour shift from yellow to red.

The labels of the present invention, namely the first label, the second label, the third or/and the fourth label, may be any detectable label known in the art. It is preferred that the first label can be discriminated from the second label and the third label. The first label, the second label, the third or/and the fourth label may be detected by a method providing a resolution down to the individual cell. In particular, the first label, the second label, the third or/and the fourth label is detected by a method independently selected from epifluorescence microscopy, flow cytometry, laser scanning techniques, time resolved fluorometry, luminescence detection, isotope detection, hyper spectral imaging scanner, Surface Plasmon Resonance and another evanescence based reading technology.

The first, the second, the third or/and the fourth label, as used herein, may be a luminescent labelling group. Many fluorophores suitable as labelling groups in the present invention are available. The labelling group may be selected to fit the filters present in the market. The labelling group may be any suitable labelling group which can be detected in a micro-organism. Preferably, the labelling group is a fluorescent labelling group. More preferably, the labelling group is selected from fluorescein, Atto-495-NSI, FAM, Atto550, Atto Rho6G, DY520XL, and DY521XL.

The labelling group may be coupled via a spacer. Many spacers are known in the art and may be applied. Using protein chemistry techniques well known in the art many ways of attaching a spacer and subsequently attaching a fluorophore are feasible. In a preferred embodiment cysteine is chosen as its primary amino group may readily be labelled with a fluorophore. Molecules with longer carbon backbones and other reactive groups well known in the art may also be chosen as linker/spacer.

It is preferred to use in the method of the present invention an oligo-nucleotide labelled with a first label being a fluorophore emitting at a predetermined wavelengths range together with a probe labelled with second, third or/and fourth fluorophore emitting at a different wavelengths range, so that the two fluorophores can be discriminated by luminescence detection. For instance, one of the fluorophors, such as Fluorescein, may emit a green signal, and the other fluorophor may emit a red signal.

In the method of the present invention, steps (a) and (c) may be performed simultaneously or separately. Preferably, steps (a) and (c) are performed simultaneously.

In the method of the present invention, steps (b) and (d) may be performed simultaneously or separately. Preferably, steps (b) and (d) are performed simultaneously.

It is also preferred that in the method of the present invention, steps (a) and (c) are performed simultaneously, and steps (b) and (d) are performed simultaneously.

Preferably, the same detection method is employed for both the identification of the micro-organism and the detection of the antibiotic resistance in the micro-organism. Said detection method may be any detection method as described herein, for example epifluorescence microscopy, flow cytometry, laser scanning devices or another detection method described herein.

It is also preferred that in the method steps (a), (b), (c) and (d) are performed simultaneously.

By simultaneously performing the steps of the present invention, in particular steps (b) and (d), the characterisation of micro-organisms in routine diagnostic procedures can be improved, as described herein. Improvement refers in particular to automatisation.

Preferably, in-situ hybridisation is combined with detection of antibiotic resistance. More preferably, FISH is combined with detection of antibiotic resistance.

In the method of the present invention identification of the micro-organism may be performed under in-situ conditions, in particular by fluorescence in-situ hybridization (FISH). Detection of antibiotic resistance by a nucleic acid, as described herein, may also be performed under in-situ conditions, in particular by fluorescence in-situ hybridization. It is preferred that identification of the micro-organism and detection of antibiotic resistance by a nucleic acid, as described herein, may also be performed under in-situ conditions, in particular by fluorescence in-situ hybridization (FISH).

FISH, as used herein, can include PNA FISH and bbFISH.

In the method of the present invention steps (a), (b), (c) or/and (d) may comprise in-situ conditions, in particular FISH. In-situ hybridisation, in particular FISH, conventionally calls for specific environments for their respective assays of the state of the art. It was therefore surprising that it was possible to
1. prepare the cells for in-situ hybridisation with pores of sufficient size to allow passage of up to 50-mer oligo-nucleotides;
2. make membrane proteins accessible for the probe;
3. maintain the integrity of both said proteins and ribosomes to allow the specific binding of the probe;
4. Find sufficient binding sites to generate a signal visible under an epifluorescence microscope, in particular under uniform conditions.

If step (c) refers to FISH, this step in particular relates to detection of antibiotic resistance by a nucleic acid according to step (c)(II), as described herein.

The method of the present invention may comprise steps to remove labelled probes which are not bound to a micro-organism. Such steps may improve the signal-to-noise ratio.

The method steps (a), (b), (c) and (d) can be performed on an automated platform. In particular, simultaneous detection of the first label and the second, the third, or/and the fourth label can be performed on a automated platform by the simultaneous or consecutive detection of the signals from the first label and the second, the third, or/and the fourth label. Detection of the signals can be performed by computer analysis of one or more microscopic images. Simultaneous detection of the first label and the second, the third, or/and the fourth label is preferred. The first label and the second, the third, or/and the fourth label are preferably different.

In the present invention, the predetermined micro-organism in its non-resistant form can be employed as a reference to determine the presence of the first label, the second label, the third label, the fourth label, or/and the modified substrate. The predetermined micro-organism in its non-resistant form may be added to the sample, or may be presented in a separate preparation. The predetermined micro-organism in its non-resistant form may carry at least one further label. Any label as described herein may be employed, provided this label is suitable for discrimination from the label employed for detection of the micro-organism or/and for identification of the antibiotic resistance, or/and other micro-organisms present in the assay of the present invention. The amount of detectable label in a predetermined micro-organism in its non-resistant form may also be provided in the form of specific values or ranges of the amount of detectable label for one or more combinations comprising (a) the micro-organism, (b) an antibiotic, and (c) at least one labelling group, for instance in the form of a data sheet. In particular, a kit of the present invention may comprise said predetermined micro-organism in its non-resistant form or/and said data sheet.

The method of the present invention may also employ the predetermined micro-organism in its resistant form as a further control, or specific values or ranges of the amount of detectable label in a predetermined micro-organism in its resistant form for one or more combinations comprising (a) the micro-organism, (b) an antibiotic, and (c) at least one labelling group, for instance in the form of a data sheet, as described above.

The micro-organism may be detected or/and identified by an increase of the amount of detectable label of at least 5%, at least 10%, at least 20%, at least 30%, at least 40%, at least 50%, at least 60%, at least 70%, at least 80%, at least 90%, at least 100%, at least 150%, or at least 200% with respect to the amount of detectable label in the predetermined micro-organism in its non-resistant form.

In the method of the present invention, the resistance of beta-lactam antibiotics can be detected by a modified or altered surface receptors or/and glycopeptides. For example, a MRSA or/and ORSA having a modified Penicillin Binding Protein (PBP2a) can be detected. In another example, a VRE, VRSA or VRCD comprising modified peptidoglycans comprising a D-alanine-D-lactate C-terminus can be detected.

In the method of the present invention, the resistance towards beta-lactam antibiotics can be detected by a mRNA. For example, a MRSA or/and ORSA expressing a mecA mRNA can be detected, which for, example, is not present in Methicillin Sensitive Staphylococcus aureus (MSSA). In another example, a VRE, VRSA or VRCD comprising a modified vanA, vanB or/and vanC mRNA can be detected.

A preferred resistance factor is the PBP2 protein (Penicillin Binding Protein) in Staphylococcus encoded by the mecA gene. In Staphylococcus resistant against beta-lactam antibiotics, the mecA gene encodes a modified PBP2 protein (PBP2′ or PBP2a) with low affinity for methicillin and many 11-lactam antibiotics, in particular with low affinity for penicillins. Thus, in a more preferred embodiment, (a) the micro-organism is a MRSA or/and ORSA strain harbouring the mecA gene, which encodes a modified PBP2 protein (PBP2′ or PBP2a) with low affinity for methicillin and many 11-lactam antibiotics, in particular with low affinity for penicillins, and (b) the antibiotic is a beta-lactam antibiotic.

In the method of the present invention, the resistance towards beta-lactam antibiotics can be detected by anti-beta-lactamase antibodies including antibodies against carbapenemases or/and NDM1. In particular, the resistance towards penicillins can be detected by anti-beta-lactamase antibodies.

In the method of the present invention, the resistance towards beta-lactam antibiotics, in particular penicillins, can be detected by compounds which are cleaved by a beta-lactamase. For example, nitrocefin changes colour from yellow to red when cleaved by a beta-lactamase.

In one particular aspect, in the method of the present invention, the resistance against a beta-lactam antibiotic, such as a penicillin, is detected by an antibody or/and a fragment thereof, specifically binding to beta-lactamase or/and PBP2a binding protein.

In yet another particular aspect, in the method of the present invention, the resistance against a beta-lactam antibiotic, such as a penicillin, is detected by a nucleic acid specifically hybridizing with an RNA encoding for beta-lactamase or/and PBP2a binding protein.

In yet another particular aspect, in the method of the present invention, the resistance against a beta-lactam antibiotic is detected by a knottin, a cystine knot protein or/and an aptamer specifically binding to beta-lactamase or/and PBP2a binding protein.

In yet another particular aspect, in the method of the present invention, the resistance against a beta-lactam antibiotic is detected by a substrate modified by beta-lactamase, wherein the substrate preferably is nitrocefin.

In the method of the present invention, the resistance towards quinolones, macrolides, ketolides, aminoglycosides or/and lincosamides can be detected by a labelled antibody directed against an efflux pump, in particular an efflux pump as described herein.

In the method of the present invention, the resistance towards quinolones, macrolides, ketolides, aminoglycosides or/and lincosamides can be detected by a mutation in mRNA, as described herein.

In the method of the present invention, the resistance towards quinolones, macrolides, ketolides, aminoglycosides or/and lincosam ides can be detected by a mutation in rRNA, in particular mutations of 23S rRNA or 16S RNA, as described herein.

In yet another particular aspect, in the method of the present invention, (a) the micro-organism is a Methicillin Resistant Staphylococcus aureus (MRSA) or/and an Oxacillin Resistant Staphylococcus aureus (ORSA), and (b) the antibiotic resistance is detected by the expression of a modified or altered Penicillin Binding Protein 2 (termed PBP2a). In this aspect MRSA or/and ORSA is discriminated from other Staphylococci, some of which can also express PBP2a. Preferably, the MRSA or/and ORSA is discriminated from Staphylococcus epidermidis, which can constitutively express PBP2a. In this aspect, the expression of PBP2a can be induced, as described herein.

In yet another particular aspect, in the method of the present invention, the micro-organism is a Methicillin Resistant Staphylococcus aureus (MRSA) or/and an Oxacillin Resistant Staphylococcus aureus (ORSA), and wherein the antibiotic resistance is detected by the expression of a mecA protein.

In yet another particular aspect, in the method of the present invention, the micro-organism is a Methicillin Resistant Staphylococcus aureus (MRSA) or/and an Oxacillin Resistant Staphylococcus aureus (ORSA), wherein the Methicillin Resistant Staphylococcus aureus (MRSA) or/and the Oxacillin Resistant Staphylococcus aureus (ORSA) are discriminated from a Methicillin Sensitive Staphylococcus aureus (MSSA) by the detection of the expression of a mecA protein.

In yet another particular aspect, in the method of the present invention, the micro-organism is selected from Vancomycin Resistant Staphylococcus aureus (VRSA), Vancomycin Resistant Staphylococcus (VRS), Vancomycin Resistant Enterococci (VRE) and Vancomycin Resistant Clostridium difficile (VRCD), and wherein the antibiotic resistance is detected by the expression of a peptide selected from vanA protein, vanB protein, vanC protein or/and modified peptidoglycans comprising a D-alanine-D-lactate C-terminus.

In yet another particular aspect, in the method of the present invention, (a) the micro-organism is selected from Gram negative bacteria, as described herein, and (b) a resistance against beta lactam antibiotics is detected. In yet another particular aspect, in the method of the present invention, (a) the micro-organism is selected from Streptococci, and (b) a resistance to macrolides, lincosamide and streptogramin (MLS) is detected.

In yet another particular aspect, in the method of the present invention, (a) the micro-organism is drug resistant Streptococcus pneumoniae (DRSP), and (b) a resistance to towards beta-lactam antibiotics and macrolides is detected.

In yet another particular aspect, in the method of the present invention, high level Aminoglycoside resistant Enterococci (HLAR) are detected.

The biological sample comprising the predetermined micro-organisms may be pretreated in order to facilitate binding of the labelled antibiotic and optionally identification of the micro-organism.

The biological sample may be heat-fixed on a carrier (for example on a slide) according to their designated labelled probes according to step (a) and (c), as described herein, for instance at about 45° C. to about 65° C., preferably at about 50° C. to about 55° C., more preferably at about 52° C.

If the micro-organism is a Gram positive bacterium, it may be perforated by a suitable buffer. Gram positive cells may be perforated with a bacteriocin or/and a detergent. In a preferred embodiment a biological detergent is employed, and a specially preferred embodiment Nisin is combined with Saponin. In addition lytic enzymes such as Lysozyme and Lysostaphin may be applied. Lytic enzymes may be balanced into the equation. If the sample is treated with ethanol, the concentration of the active ingredients may be balanced with respect to their subsequent treatment in ethanol. In a more preferred embodiment the concentration of Lysozyme, Lysostaphin, Nisin and Saponin is balanced to cover all Gram positive organisms. An example of a Gram Positive Perforation Buffer is given in Table 1. It is contemplated that variations of the amounts and concentrations, and application temperatures and incubation times are within the skill in the art.

If the micro-organism is a yeast or a mould, it may be perforated by a suitable buffer. Surprisingly it was found that the cell walls of yeasts and moulds did not form reproducible pores when treated following procedures well known in the art. These procedures frequently rendered both false positive and false negative results. A reliable solution is a preferred buffer comprising a combination of a peptide antibiotic, detergent, complexing agent, and reducing agent. A more preferred buffer comprises the combination of a mono-valent salt generating a specific osmotic pressure, a bacteriocin, a combination of biological and synthetic detergents, a complexing agent for divalent cations, and an agent capable of reducing disulfide bridges. A further surprising improvement was achieved by adding proteolytic enzymes specific for prokaryotes. In an even more preferred buffer, Saponin, SDS, Nisin, EDTA, DTT were combined with Lysozyme and a salt, for instance in a concentration of about 150 to about 250 mM, more preferably about 200 to about 230 mM, most preferably about 215 mM. An example of Yeast Perforation Buffer is given in Table 1. It is contemplated that variations of the amounts and concentrations, and application temperatures and incubation are within the skill in the art.

In yet another preferred embodiment, the method of the present invention is a diagnostic method. As described herein, the method of the present invention detects a micro-organism in a biological sample. The method of the present invention, in particular the diagnostic method of the present invention, is preferably an in-vitro method.

Yet another subject of the present invention is the use of

(a) a first nucleic acid capable of selectively hybridizing with a nucleic acid in the micro-organism, and
(b) at least one probe for detection of an antibiotic resistance, wherein said at least one probe is selected from
(i) antibodies and fragments thereof, wherein the antibodies and fragments thereof are capable of selectively binding to a resistance factor, and wherein the antibody and fragments thereof are labelled with a second label,
(ii) second nucleic acids capable of hybridizing with an RNA coding for a resistance factor, wherein the second nucleic acid is labelled with a third label,
(iii) knottins, cystine-knot proteins or/and aptamers capable of selectively binding to a resistance factor, wherein the knottins, cystine-knot proteins or/and aptamers are labelled with a fourth label, and
(iv) substrates which can be modified by a resistance factor, for the detection of an antibiotic resistance in a predetermined micro-organism in a biological sample.

Yet another subject of the present invention is a combination comprising

(a) a first nucleic acid capable of selectively hybridizing with a nucleic acid in the micro-organism, and
(b) at least one probe for detection of an antibiotic resistance, wherein said at least one probe is selected from
(i) antibodies and fragments thereof, wherein the antibodies and fragments thereof are capable of selectively binding to a resistance factor, and wherein the antibody and fragments thereof are labelled with a second label,
(ii) second nucleic acids capable of hybridizing with an RNA coding for a resistance factor, wherein the second nucleic acid is labelled with a third label,
(iii) knottins, cystine-knot proteins or/and aptamers capable of selectively binding to a resistance factor, wherein the knottins, cystine-knot proteins or/and aptamers are labelled with a fourth label, and
(iv) substrates which can be modified by a resistance factor,
for use in the detection of an antibiotic resistance in a predetermined micro-organism in particular in a biological sample.

Yet another subject of the present invention is use of a combination comprising

(a) a first nucleic acid capable of selectively hybridizing with a nucleic acid in the micro-organism, and
(b) at least one probe for detection of an antibiotic resistance, wherein said at least one probe is selected from
(i) antibodies and fragments thereof, wherein the antibodies and fragments thereof are capable of selectively binding to a resistance factor, and wherein the antibody and fragments thereof are labelled with a second label,
(ii) second nucleic acids capable of hybridizing with an RNA coding for a resistance factor, wherein the second nucleic acid is labelled with a third label,
(iii) knottins, cystine-knot proteins or/and aptamers capable of selectively binding to a resistance factor, wherein the knottins, cystine-knot proteins or/and aptamers are labelled with a fourth label, and
(iv) substrates which can be modified by a resistance factor,
for preparation of a kit for the detection of an antibiotic resistance in a predetermined micro-organism in particular in a biological sample.

The present invention is further illustrated by the following tables.

Table 1 describes the composition of perforation buffers employed in the present invention.

Table 2: Antibiotic resistance due to mutations on the 23S rRNA.

Table 3: Antibiotic resistance mechanism in clinically relevant micro-organisms and alteration in the amount of antibiotics in resistant micro-organisms. The alteration of the amount of detectable labelled antibiotics in micro-organisms in its resistant form is determined relative to its non-resistant form. The amount is expressed in % change of fluorescence (decrease and increase, respectively) of an antibiotic which carries a fluorescent label.

### REFERENCES

1. Chambers, H. F. (1997). Methicillin resistance in staphylococci: molecular and biochemical basis and clinical implications. Clinical Microbiology Reviews 10, 781-791.
2. Swenson, J. M., Williams, P., Killgore, G. et al. (2001). Performance of eight methods, including two new methods for detection of oxacillin resistance in a challenge set of Staphylococcus aureus organisms. Journal of Clinical Microbiology 39, 3785-8
3. Van Leeuwen, W. B., Van Pelt, C., Luijendijk, A. et al. (1999). Rapid detection of methicillin resistance in Staphylococcus aureus isolates by the MRSA-screen latex agglutination test. Journal of Clinical Microbiology 37, 3029-30.
4. Louie, L., Majury, A., Goodfellow, J. et al. (2001). Evaluation of a latex agglutination test (MRSA-Screen) for detection of oxacillin resistance in coagulase-negative staphylococci. Journal of Clinical Microbiology 39, 4149-51
5. Skov, R., Smyth, R., Clausen, M. et al. (2003). Evaluation of cefoxitin 30 μg disc on Iso-Sensitest agar for detection of methicillin-resistant Staphylococcus aureus. Journal of Antimicrobial Chemotherapy 52, 204-7
6. Felten, A., Grandry, B., Lagrange, P. H. et al. (2002). Evaluation of three techniques for detection of low-level methicillin-resistant S. aureus (MRSA): a disc diffusion method with cefoxitin and moxalactam, the Vitek 2 system, and the MRSA-screen latex agglutination test. Journal of Clinical Microbiology 40, 2766-71.
7. Cauwelier, B., Gordts, B., Descheemaecker, P. et al. (2004). Evaluation of a disk diffusion method with cefoxitin (30 μg) for detection of methicillin-resistant Staphylococcus aureus. European Journal of Clinical Microbiology and Infectious Diseases 23, 389-92.
8. Kluytmans, J., Van Griethuysen, A., Willemse, P. et al. (2002). Performance of CHROM agar selective medium and oxacillin resistance screening agar base for identifying Staphylococcus aureus and detecting methicillin resistance. Journal of Clinical Microbiology 40, 2480-2.
9. Louie, L., Matsumura, S. 0., Choi, E. et al. (2000). Evaluation of three rapid methods for detection of methicillin resistance in S. aureus. Journal of Clinical Microbiology 38, 2170-3.
10. National Committee for Clinical Laboratory Standards. (2003). Methods for Dilution Antimicrobial Susceptibility Tests for Bacteria that Grow Aerobically: Approved Standards M7-A6 and M100-513. NCCLS, Wayne, Pa., USA.

TABLE 1

| Gram-Positive Perforation | 50 | μg/ml | Saponin |
|---|---|---|---|
| Buffer- | 5 | μg/ml | Nisin |
| | 20 | mM | Tris pH 8 |
| | 100 | μg/m | Lysozym |
| | 50 | μg/ml | Lysostaphin |
| | | | H₂O |
| | Working soln. | | |
| Yeast Perforation Buffer | 500 | μg/ml | Saponin |
| | 10 | μg/ml | Nisin |
| | 50 | mM | Tris pH 8.3 |
| | 215 | mM | NaCl |

| 0.1% | | SDS |
|---|---|---|
| 5 | mM | EDTA |
| 10 | mM | DTT |
| 100 | μg/ml | Lysozyme |
| | | H₂O |

TABLE 2
Compilation of antibiotic resistance due to mutations on the 23S rRNA

| Type | | | | | |
|---|---|---|---|---|---|
| of | | | | | |
| RNA | Position | Alteration(s) | Phenotype | Organism | Reference |
| 23S | 2032 | AG to GA | Clr/Azm/Ery-R | Helicobacter | Húlten, K., A. Gibreel, O. Sköld, and L. Engstrand. |
| | | | | pylori | 1997. Macrolide resistance in Helicobacter pylori: |
| | | | | | mechanism and stability in strains from |
| | | | | | clarithromycin-treated patients. Antimicrob. Agents |
| | | | | | Chemother. 41: 2550-2553. |
| 23S | 2058 | A to C | Clr-R | Helicobacter | Stone, G. G., D. Shortridge, R. K. Flamm, J. Versalovic, |
| | | | | pylori | J. Beyer, K. Idler, L. Zulawinski, and S. K. Tanaka. |
| | | | | | 1996. Identification of a 23S rRNA gene |
| | | | | | mutation in clarithromycin-resistant Helicobacter |
| | | | | | pylori. Helicobacter. 1: 227-228. |
| 23S | 2058 | A to C | Mac-R, Lin-R | Helicobacter | Occhialini, A., M. Urdaci, F. Doucet-Populaire, |
| | | | | pylori | C. M. Bébéar, H. Lamouliatte, and F. Mégraud. 1997. |
| | | | | | Macrolide resistance in Helicobacter pylori: rapid |
| | | | | | detection of point mutations and assays of macrolide |
| | | | | | binding to ribosomes. Antimicrob. Agents Chemothe |
| 23S | 2058 | A to C | MLSB-R | Helicobacter | Wang, G., and D. E. Taylor. 1998. Site-specific |
| | | | | pylori | mutations in the 23S rRNA gene of Helicobacter |
| | | | | | pylori confer two types of resistance to macrolide- |
| | | | | | lincosamide-streptogramin B antibiotics. Antimicrob. |
| | | | | | Agents Chemother. 42: 1952-1958. |
| 23S | 2058 | A to C | Cla-R | Helicobacter | Debets-Ossenkopp, Y. J., A. B. Brinkman, E. J. Kuipers, |
| | | | | pylori | C. M. Vandenbroucke-Grauls, and J. G. Kusters. |
| | | | | | 1998. Explaining the bias in the 23S rRNA |
| | | | | | gene mutations associated with clarithromycin |
| | | | | | resistance in clinical isolates of Helicobacter pylori. |
| | | | | | Antimi |
| 23S | 2058 | A to G | Cla-R | Helicobacter | Versalovic, J., D. Shortridge, K. Kibler, M. V. Griffy, |
| | | | | pylori | J. Beyer, R. K. Flamm, S. K. Tanaka. D. Y. Graham, and |
| | | | | | M. F. Go. 1996. Mutations in 23S rRNA are |
| | | | | | associated with clarithromycin resistance in |
| | | | | | Helicobacter pylori. Antimicrob. Agents Chemother. |
| | | | | | 40:4 |
| 23S | 2058 | A to G | Mac-R, Lin-R | Helicobacter | Occhialini, A., M. Urdaci, F. Doucet-Populaire, |
| | | | | pylori | C. M. Bébéar, H. Lamouliatte, and F. Mégraud. 1997. |
| | | | | | Macrolide resistance in Helicobacter pylori: rapid |
| | | | | | detection of point mutations and assays of macrolide |
| | | | | | binding to ribosomes. Antimicrob. Agents Chemothe |
| 23S | 2058 | A to G | MLSB-R | Helicobacter | Wang, G., and D. E. Taylor. 1998. Site-specific |
| | | | | pylori | mutations in the 23S rRNA gene of Helicobacter |
| | | | | | pylori confer two types of resistance to macrolide- |
| | | | | | lincosamide-streptogramin B antibiotics. Antimicrob. |
| | | | | | Agents Chemother. 42: 1952-1958. |
| 23S | 2058 | A to G | Cla-R | Helicobacter | Debets-Ossenkopp, Y. J., A. B. Brinkman, E. J. Kuipers, |
| | | | | pylori | C. M. Vandenbroucke-Grauls, and J. G. Kusters. |
| | | | | | 1998. Explaining the bias in the 23S rRNA |
| | | | | | gene mutations associated with clarithromycin |
| | | | | | resistance in clinical isolates of Helicobacter pylori. |
| | | | | | Antimi |
| 23S | 2058 | A to U | MLSB-R | Helicobacter | Wang, G., and D. E. Taylor. 1998. Site-specific |
| | | | | pylori | mutations in the 23S rRNA gene of Helicobacter |
| | | | | | pylori confer two types of resistance to macrolide- |
| | | | | | lincosamide-streptogramin B antibiotics. Antimicrob. |
| | | | | | Agents Chemother. 42: 1952-1958. |
| 23S | 2058 | A to U | Cla-R | Helicobacter | Debets-Ossenkopp, Y. J., A. B. Brinkman, E. J. Kuipers, |
| | | | | pylori | C. M. Vandenbroucke-Grauls, and J. G. Kusters. |
| | | | | | 1998. Explaining the bias in the 23S rRNA |
| | | | | | gene mutations associated with clarithromycin |
| | | | | | resistance in clinical isolates of Helicobacter pylori. |
| | | | | | Antimi |
| 23S | 2059 | A to C | Mac-R, Lin-R, | Helicobacter | Wang, G., and D. E. Taylor. 1998. Site-specific |
| | | | SB-S | pylori | mutations in the 23S rRNA gene of Helicobacter |
| | | | | | pylori confer two types of resistance to macrolide- |
| | | | | | lincosamide-streptogramin B antibiotics. Antimicrob. |
| | | | | | Agents Chemother. 42: 1952-1958. |
| 23S | 2059 | A to C | Clr-R | Helicobacter | Debets-Ossenkopp, Y. J., A. B. Brinkman, E. J. Kuipers, |
| | | | | pylori | C. M. Vandenbroucke-Grauls, and J. G. Kusters. |
| | | | | | 1998. Explaining the bias in the 23S rRNA |
| | | | | | gene mutations associated with clarithromycin |
| | | | | | resistance in clinical isolates of Helicobacter pylori. |
| | | | | | Antimi |
| 23S | 2059 | A to G | Clr-R | Helicobacter | Versalovic, J., D. Shortridge, K. Kibler, M. V. Griffy, |
| | | | | pylori | J. Beyer, R. K. Flamm, S. K. Tanaka. D. Y. Graham, and |
| | | | | | M. F. Go. 1996. Mutations in 23S rRNA are |
| | | | | | associated with clarithromycin resistance in |
| | | | | | Helicobacter pylori. Antimicrob. Agents Chemother. |
| | | | | | 40:4 |
| 23S | 2059 | A to G | Mac-R, Lin-R | Helicobacter | Occhialini, A., M. Urdaci, F. Doucet-Populaire, |
| | | | | pylori | C. M. Bébéar, H. Lamouliatte, and F. Mégraud. 1997. |
| | | | | | Macrolide resistance in Helicobacter pylori: rapid |
| | | | | | detection of point mutations and assays of macrolide |
| | | | | | binding to ribosomes. Antimicrob. Agents Chemothe |
| 23S | 2059 | A to G | Mac-R, Lin-R, | Helicobacter | Wang, G., and D. E. Taylor. 1998. Site-specific |
| | | | SB-S | pylori | mutations in the 23S rRNA gene of Helicobacter |
| | | | | | pylori confer two types of resistance to macrolide- |
| | | | | | lincosamide-streptogramin B antibiotics. Antimicrob. |
| | | | | | Agents Chemother. 42: 1952-1958. |
| 23S | 2059 | A to G | Cla-R | Helicobacter | Debets-Ossenkopp. Y. J., A. B. Brinkman E. J. Kuipers, |
| | | | | pylori | C. M. Vandenbroucke-Grauls, and J. G. Kusters. |
| | | | | | 1998. Explaining the bias in the 23S rRNA |
| | | | | | gene mutations associated with clarithromycin |
| | | | | | resistance in clinical isolates of Helicobacter pylori. |
| | | | | | Antimi |
| 23S | 754 | “U to A” | Resistant to low | E. coli | Xiong L, Shah S, Mauvais P, Mankin AS. |
| | | | concentrations of ketolide | | 1999. A ketolide resistance mutation in |
| | | | HMR3647; resistant to | | domain II of 23S rRNA reveals the |
| | | | erythromycin b. | | proximity of hairpin 35 to the peptidyl |
| | | | | | transferase center. Molecular |
| | | | | | Microbiology 31 (2): 633-639. |
| 23S | 754 | “U to A” | Confers macrolide and | E. coli | Hansen L. H., Mauvais P, Douthwaite S. |
| | | | ketolide resistance. | | 1999. The macrolide-ketolide antibiotic |
| | | | | | binding site is formed by structures in |
| | | | | | domain II and V of 23S ribosomal RNA. |
| | | | | | Molecular Microbiology 31 (2): 623-631. |
| 23S | 754 | U to A | Ery-LR, Tel-LR | Escherichia | Xiong, L., S. Shah, P. Mauvais, and A. S. Mankin. |
| | | | | coli | 1999. A ketolide resistance |
| | | | | | mutation in domain II of 23S rRNA reveals |
| | | | | | the proximity of hairpin 35 to the peptidyl |
| | | | | | transferase centre. Mol. Microbiol. |
| | | | | | 31: 633-639. |
| 23S | 1005 | C to G | Slow growth under natural | E. coli | 1) Rosendahl, G. and Douthwaite, S. |
| | | | promoter; (with 2058G and | | (1995) Nucleic Acids Res. 23, 2396-2403. |
| | | | erythromycin) severe growth | | 2) Rosendahl, G., Hansen, L. H., and |
| | | | retardation. A | | Douthwaite, S. (1995) J. Mol. Biol. 249, |
| | | | | | 59-68. |
| 23S | 1005 | C to G | Slow growth under pL | E. coli | 1) Rosendahl, G. and Douthwaite, S. |
| | | | promoter; (with 2058G and | | (1995) Nucleic Acids Res. 23, 2396-2403. |
| | | | erythromycin) Erys. a Double | | 2) Rosendahl, G., Hansen, L. H., and |
| | | | mutant (C1005G/C1006U) | | Douthwaite, S. (1995) J. Mol. Biol. 249, |
| | | | | | 59-68. |
| 23S | 1006 | C to U | Slow growth under pL | E. coli | 1) Rosendahl, G. and Douthwaite, S. |
| | | | promoter; (with 2058G and | | (1995) Nucleic Acids Res. 23, 2396-2403. |
| | | | erythromycin) Erys. a Double | | 2) Rosendahl, G., Hansen, L. H., and |
| | | | mutant (C1005G/C1006U) | | Douthwaite, S. (1995) J. Mol. Biol. 249, |
| | | | | | 59-68. |
| 23S | 1006 | C to U | Lethal under natural | E. coli | 1) Rosendahl, G. and Douthwaite, S. |
| | | | promoter; under pL | | (1995) Nucleic Acids Res. 23, 2396-2403. |
| | | | promoter; (with 2058G and | | 2) Rosendahl, G., Hansen, L. H., and |
| | | | erythormycin) Erys. A | | Douthwaite, S. (1995) J. Mol. Biol. 249, |
| | | | | | 59-68. |
| 23S | 1056 | G to A | Binding of both L11 and | E. coli | Ryan, P. C. and Draper, D. E. (1991) Proc. |
| | | | thiostrepton is weakened in | | Natl. Acad. Sci. USA 88, 6308-6312. |
| | | | RNA fragments. B | | |
| 23S | 1056 | G to A | Stoichiometric L11 binding.b | E. coli | 1) Douthwaite, S. and Aagaard, C. (1993) |
| | | | (with 2058G and | | J. Mol. Biol. 232, 725-731. 2) Rosendahl, G. |
| | | | erythromycin) Reduced | | and Douthwaite, S. (1995) Nucleic |
| | | | growth rate. a | | Acids Res. 23, 2396-2403. |
| 23S | 1056 | G to C | Binding of thiostrepton is | E. coli | Ryan, P. C. and Draper, D. E. (1991) Proc. |
| | | | weakened in RNA | | Natl. Acad. Sci. USA 88, 6308-6312. |
| | | | fragments. B | | |
| 23S | 1064 | C to U | Stoichiometric L11 binding. b | E. coli | 1) Douthwaite, S. and Aagaard, C. (1993) |
| | | | (with 2058G and | | J. Mol. Biol. 232, 725-731. 2) Rosendahl, G. |
| | | | erythromycin) Reduced | | and Douthwaite, S. (1995) Nucleic |
| | | | growth rate. a | | Acids Res. 23, 2396-2403. |
| 23S | 1067 | A to U | Normal growth | E. coli | 1) Spahn, C., Remme, J., Schafer, M. and |
| | | | | | Nierhaus, K. (1996). J. Biol. Chem. 271: |
| | | | | | 32849-32856. 2) Spahn, C., Remme, J., |
| | | | | | Schafer, M. and Nierhaus, K. (1996). J. |
| | | | | | Biol. Chem. 271: 32857-32862. |
| 23S | 1067 | A to G | Thiostrepton resistance in | Halobacterium | Hummel, H., and A. Bock. (1987) |
| | | | Halobacterium sp. | | Biochimie 69: 857-861. |
| 23S | 1067 | A to U | Thiostrepton resistance in | Halobacterium | Hummel, H., and A. Bock. (1987) |
| | | | Halobacterium sp. | | Biochimie 69: 857-861. |
| 23S | 1067 | A to U | A to C or U confers high | E. coli | 1) Thompson, J. and Cundliffe, E. (1991) |
| | | | level resistance to | | Biochimie 73: 1131-1135. 2) Thompson, J., |
| | | | thiostrepton, whereas A to G | | Cundliffe, E. and Dahlberg, A. E. (1988) |
| | | | confers intermediate level | | J. Mol. Biol. 203: 457-465. 3) Lewicki, B. T. U., |
| | | | resistance; drug binding | | Margus, T., Remme, J. and |
| | | | affinity is reduced similarly. | | Nierhaus, K. H. (1993) J. Mol. Biol. 231, |
| | | | a, b Expression by host RNA | | 581-593. 4) LAST |
| | | | polymerase results in | | |
| | | | formation of active ribosomal | | |
| | | | subunits in vivo. A | | |
| 23S | 1067 | A to C | A to C or U confers high | E. coli | 1) Thompson, J. and Cundliffe, E. (1991) |
| | | | level resistance to | | Biochimie 73: 1131-1135. 2) Thompson, J., |
| | | | thiostrepton, whereas A to G | | Cundliffe, E. and Dahlberg, A. E. (1988) |
| | | | confers intermediate level | | J. Mol. Biol. 203: 457-465. 3) Lewicki, B. T. U., |
| | | | resistance; drug binding | | Margus, T., Remme, J. and |
| | | | affinity is reduced similarly. | | Nierhaus, K. H. (1993) J. Mol. Biol. 231, |
| | | | a, b Expression by host RNA | | 581-593. 4) LAST |
| | | | polymerase results in | | |
| | | | formation of active ribosomal | | |
| | | | subunits in vivo. A | | |
| 23S | 1067 | A to G | A to C or U confers high | E. coli | 1) Thompson, J. and Cundliffe, E. (1991) |
| | | | level resistance to | | Biochimie 73: 1131-1135. 2) Thompson, J., |
| | | | thiostrepton, whereas A to G | | Cundliffe, E. and Dahlberg, A. E. (1988) |
| | | | confers intermediate level | | J. Mol. Biol. 203: 457-465. 3) Lewicki, B. T. U., |
| | | | resistance; drug binding | | Margus, T., Remme, J. and |
| | | | affinity is reduced similarly. | | Nierhaus, K. H. (1993) J. Mol. Biol. 231, |
| | | | a, b Expression by host RNA | | 581-593. 4) LAST |
| | | | polymerase results in | | |
| | | | formation of active ribosomal | | |
| | | | subunits in vivo. A | | |
| 23S | 1067 | “A to U” | Constituted 30% of the total | E. coli | Liiv A, Remme J. 1998. Base-pairing of |
| | | | 23S rRNA pool in the | | 23S rRNA ends is essential for ribosomal |
| | | | ribosomes; exhibited 30% | | large subunit assembly. J. Mol. Biol. 285: |
| | | | thiostrepton resistance in | | 965-975. |
| | | | poly (U) translation b. | | |
| 23S | 1068 | G to A | Reduced L11 binding. b (with | E. coli | 1) Ròsendahl, G. and Douthwaite, S. |
| | | | 2058G) Lethal when | | (1995) Nucleic Acids Res. 23, 2396-2403. |
| | | | expressed from rrnB or pL | | 2) Douthwaite, S. and Aagaard, C. |
| | | | promotor in presence of | | (1993) J. Mol. Biol. 232, 725-731. |
| | | | erythromycin. A | | |
| 23S | 1068 | G to A | Suppression of 1068A; | E. coli | Rosendahl, G. and Douthwaite, S. |
| | | | lethality only in absence of | | (1995) Nucleic Acids Res. 23, 2396-2403. |
| | | | erythromycin. a Double mutant | | |
| | | | (G1068A/G1099A) | | |
| 23S | 1072 | C to U | Lethal when expressed from | E. coli | Rosendahl, G. and Douthwaite, S. |
| | | | rrnB or pL promotor in | | (1995) Nucleic Acids Res. 23, 2396-2403. |
| | | | presence of erythromycin. a | | |
| 23S | 1137 | G to A | With 2058G and erythromycin, | E. coli | Rosendahl, G., Hansen, L. H., and |
| | | | lethal when expressed from rrnB | | Douthwaite, S. (1995) J. Mol. Biol. |
| | | | promoter. | | 249, 59-68. |
| 23S | 1137 | G to A | Restores normal growth under pL | E. coli | Rosendahl, G., Hansen, L. H., and |
| | | | promotor; (With 2058G and | | Douthwaite, S. (1995) J. Mol. Biol. |
| | | | erythromycin) Eryr. Double | | 249, 59-68. |
| | | | mutant (G1137A/C1006U) | | |
| 23S | 1137 | G to A | With 2058G and erythromycin, | E. coli | Rosendahl, G., Hansen, L. H., and |
| | | | lethal when expressed from rrnB | | Douthwaite, S. (1995) J. Mol. Biol. 249, |
| | | | promoter. Double mutant | | 59-68. |
| | | | (G1137A/G1138C) | | |
| 23S | 1138 | G to C | With 2058G and erythromycin, | E. coli | Rosendahl, G., Hansen, L. H., and |
| | | | lethal when expressed from rrnB | | Douthwaite, S. (1995) J. Mol. Biol. 249, |
| | | | promoter. | | 59-68. |
| 23S | 1138 | G to C | With 2058G and erythromycin, | E. coli | Rosendahl, G., Hansen, L. H., and |
| | | | lethal when expressed from rrnB | | Douthwaite, S. (1995) J. Mol. Biol. 249, |
| | | | promoter. Double mutant | | 59-68. |
| | | | (G1138C/G1137A) | | |
| 23S | 1207 | C to U | Erythromycin resistant. a Double | E. coli | Dam, M., Douthwaite, S., Tenson, T. |
| | | | mutant (C1207U/C1243U) | | and Mankin, A. S. (1996) J. Mol. Biol. |
| | | | | | 259, 1-6. |
| 23S | 1208 | C to U | Erythromycin resistant. a Double | E. coli | Dam, M., Douthwaite, S., Tenson, T. |
| | | | mutant (C1208U/C1243U) | | and Mankin, A. S. (1996) J. Mol. Biol. |
| | | | | | 259, 1-6. |
| 23S | 1211 | C to U | Erythromycin sensitive. a Double | E. coli | Dam, M., Douthwaite, S., Tenson, T. |
| | | | mutant (C1211U/C1208U) | | and Mankin, A. S. (1996) J. Mol. Biol. |
| | | | | | 259, 1-6. |
| 23S | 1220 | G to A | Erythromycin resistant. a Double | E. coli | Dam, M., Douthwaite, S., Tenson, T. |
| | | | mutant (G1220A/G1239A) | | and Mankin, A. S. (1996) J. Mol. Biol. |
| | | | | | 259, 1-6. |
| 23S | 1221 | C to U | Erythromycin resistant. a Double | E. coli | Dam, M., Douthwaite, S., Tenson, T. |
| | | | mutant (C1221U/C1229U) | | and Mankin, A. S. (1996) J. Mol. Biol. |
| | | | | | 259, 1-6. |
| 23S | 1221 | C to U | Erythromycin resistant. a Double | E. coli | Dam, M., Douthwaite, S., Tenson, T. |
| | | | mutant (C1221U/C1233U) | | and Mankin, A. S. (1996) J. Mol. Biol. |
| | | | | | 259, 1-6. |
| 23S | 1230 | 1230 | Erythromycin sensitive. a Double | E. coli | Douthwaite, S., Powers, T., Lee, J. Y., |
| | | | deletion (1230/1231) | | and Noller, H. F. (1989) J. Mol. Biol. |
| | | | | | 209, 655-665. |
| 23S | 1231 | 1231 | Erythromycin sensitive. a Double | E. coli | Douthwaite, S., Powers, T., Lee, J. Y., |
| | | | deletion (1231/1230) | | and Noller, H. F. (1989) J. Mol. Biol. |
| | | | | | 209, 655-665. |
| 23S | 1232 | G to A | Erythromycin sensitive. a Double | E. coli | Dam, M., Douthwaite, S., Tenson, T. |
| | | | mutant (G1232A/G1238A) | | and Mankin, A. S. (1996) J. Mol. Biol. |
| | | | | | 259, 1-6. |
| 23S | 1233 | C to U | Erythromycin sensitive. a | E. coli | Dam, M., Douthwaite, S., Tenson, T. |
| | | | | | and Mankin, A. S. (1996) J. Mol. Biol. |
| | | | | | 259, 1-6. |
| 23S | 1234 | “del1234/ | Erythromycin sensitive. a Double | E. coli | Douthwaite, S., Powers, T., Lee, J. Y., |
| | | del1235” | mutant (U1234C/del1235) | | and Noller, H. F. (1989) J. Mol. Biol. |
| | | | | | 209, 655-665. |
| 23S | 1234 | U to C | Erythromycin sensitive. a | E. coli | Douthwaite, S., Powers, T., Lee, J. Y., |
| | | | | | and Noller, H. F. (1989) J. Mol. Biol. 209, |
| | | | | | 655-665. |
| 23S | 1243 | C to U | Erythromycin resistant. a Double | E. coli | Douthwaite, S., Powers, T., Lee, J. Y., |
| | | | mutant (C1243U/C1208U) | | and Noller, H. F. (1989) J. Mol. Biol. 209, |
| | | | | | 655-665. |
| 23S | 1243 | C to U | Erythromycin resistant. a Double | E. coli | Douthwaite, S., Powers, T., Lee, J. Y., |
| | | | mutant (C1243U/C1221U) | | and Noller, H. F. (1989) J. Mol. Biol. 209, |
| | | | | | 655-665. |
| 23S | 1243 | “C to U” | Erythromycin resistant. a Double | E. coli | Douthwaite, S., Powers, T., Lee, J. Y., |
| | | | mutant (C1243U/C1207). | | and Noller, H. F. (1989) J. Mol. Biol. 209, |
| | | | | | 655-665. |
| 23S | 1262 | A to G | With erythromycin; lethal | E. coli | Aagaard, C., and Douthwaite, S. (1994) |
| | | | | | Proc. Natl. Acad. Sci. USA 91, 2989-2993. |
| 23S | 1262 | A to C | With erythromycin; lethal | E. coli | Aagaard, C., and Douthwaite, S. (1994) |
| | | | | | Proc. Natl. Acad. Sci. USA 91, 2989-2993. |
| 23S | 1262 | A to U | With erythromycin; reduced | E. coli | Aagaard, C., and Douthwaite, S. (1994) |
| | | | growth rate | | Proc. Natl. Acad. Sci. USA 91, 2989-2993. |
| 23S | 1262 | A to C | With erythromycin; reduced | E. coli | Aagaard, C., and Douthwaite, S. (1994) |
| | | | growth rate Double mutant | | Proc. Natl. Acad. Sci. USA 91, 2989-2993. |
| | | | (A1262C/U2017G) | | |
| 23S | 1262 | A to G | Suppression of growth effects; | E. coli | Aagaard, C., and Douthwaite, S. (1994) |
| | | | Wild-type growth on | | Proc. Natl. Acad. Sci. USA 91, 2989-2993. |
| | | | erythromycin Double mutant | | |
| | | | (A1262G/U2017C) | | |
| 23S | 1262 | A to U | Suppression of growth effects; | E. coli | Aagaard, C., and Douthwaite, S. (1994) |
| | | | Wild-type growth on | | Proc. Natl. Acad. Sci. USA 91, 2989-2993. |
| | | | erythromycin Double mutant | | |
| | | | (A1262U/U2017A) | | |
| 23S | 1262 | A to U | With erythromycin; reduced | E. coli | Aagaard, C., and Douthwaite, S. (1994) |
| | | | growth rate Double mutant | | Proc. Natl. Acad. Sci. USA 91, 2989-2993. |
| | | | (A1262U/U2017G) | | |
| 23S | 1423 | G to A | Suppressed requirement for | E. coli | O'Connor, M., Brunelli, C. A., Firpo, M. A., |
| | | | 4.5S RNA in translation of | | Gregory, S. T., Lieberman, K. R., |
| | | | natural mRNAs by cell extracts. c | | Lodmell, J. S., Moine, H., Van Ryk, D. I. |
| | | | | | and Dahlberg, A. E. (1995) Biochem. |
| | | | | | Cell Biology 73, 859-868. |
| 23S | 1698 | A to G | Suppresses 2555 mutations | E. coli | O'Connor & Dahlberg, unpublished |
| 23S | 2017 | “U to G” | Reduced growth | E. coli | Aaagard, C. and Douthwaite, S. (1994) Proc. |
| | | | rate on | | Natl. Acad. Sci. USA 91, 2989-2993. |
| | | | eyrthomycin. | | |
| 23S | 2017 | “U to C” | Reduced growth | E. coli | Aaagard, C. and Douthwaite, S. (1994) Proc. |
| | | | rate of | | Natl. Acad. Sci. USA 91, 2989-2993. |
| | | | erythromycin. | | |
| 23S | 2017 | “U to A” | Reduced growth | E. coli | Aaagard, C. and Douthwaite, S. (1994) Proc. |
| | | | rate of | | Natl. Acad. Sci. USA 91, 2989-2993. |
| | | | erythromycin. | | |
| 23S | 2017 | “U to C” | Reduced growth | E. coli | Aaagard, C. and Douthwaite, S. (1994) Proc. |
| | | | rate on | | Natl. Acad. Sci. USA 91, 2989-2993. |
| | | | erythromycin. | | |
| | | | Double mutation | | |
| | | | (U2017C/A1262G) | | |
| 23S | 2017 | “U to G” | Reduced growth | E. coli | Aaagard, C. and Douthwaite, S. (1994) Proc. |
| | | | rate on | | Natl. Acad. Sci. USA 91, 2989-2993. |
| | | | erythromycin. | | |
| | | | Double mutation | | |
| | | | (U2017G/A1262C) | | |
| 23S | 2017 | “U to G” | Reduced growth | E. coli | Aaagard, C. and Douthwaite, S. (1994) Proc. |
| | | | rate on | | Natl. Acad. Sci. USA 91, 2989-2993. |
| | | | erythromycin. | | |
| | | | Double mutation | | |
| | | | (U2017G/A1262U) | | |
| 23S | 2032 | “G to A” | Lincomycin | Tobbaco | Cseplö, A., Etzold, T., Schell, J., and Schreier, P. H. |
| | | | resistance. | chloroplasts | (1988) Mol. Genet. 214, 295-299. |
| 23S | 2032 | “G to A” | EryS, Cds, Cms. | E. coli | 1. Douthwaite, S. (1992) J. Bacteriol. 174, |
| | | | Double mutation | | 1333-1338. 2. Aaagard, C. and Douthwaite, S. |
| | | | (G2032A/A2058G) | | (1994) Proc. Natl. Acad. Sci. USA 91, |
| | | | | | 2989-2993. 3. Vester, B., Hansen, L. H., and |
| | | | | | Douthwaite, S. (1995) RNA 1, 501-509. |
| 23S | 2032 | “G to A” | Eryhs, Cds, Cms. | E. coli | 1. Douthwaite, S. (1992) J. Bacteriol. 174, |
| | | | Double mutation | | 1333-1338. 2. Aaagard, C. and Douthwaite, S. |
| | | | (G2032A/A2058U) | | (1994) Proc. Natl. Acad. Sci. USA 91, |
| | | | | | 2989-2993. 3. Vester, B., Hansen, L. H., and |
| | | | | | Douthwaite, S. (1995) RNA 1, 501-509. |
| 23S | 2032 | “G to A” | Eryr, Cdr, Cmr. | E. coli | 1. Douthwaite, S. (1992) J. Bacteriol. 174, |
| | | | Double mutation | | 1333-1338. 2. Aaagard, C. and Douthwaite, S. |
| | | | (G2032A/G2057A) | | (1994) Proc. Natl. Acad. Sci. USA 91, |
| | | | | | 2989-2993. 3. Vester, B., Hansen, L. H., and |
| | | | | | Douthwaite, S. (1995) RNA 1, 501-509. |
| 23S | 2032 | AG to GA | Clr/Azm/Ery-R | Helicobacter | Húlten, K., A. Gibreel, O. Sköld, and L. Engstrand. |
| | | | | pylori | 1997. Macrolide resistance in |
| | | | | | Helicobacter pylori: mechanism and stability |
| | | | | | in strains from clarithromycin-treated patients. |
| | | | | | Antimicrob. Agents Chemother. 41: 2550-2553. |
| 23S | 2051 | “del A” | Prevents ErmE | E. coli | Vester B, Nielsen AK, Hansen LH, Douthwaite S. |
| | | | methylation. c | | 1998. ErmE Methyltransferase Recognition |
| | | | | | Elements in RNA Substrates. J. Mol. Biol. |
| | | | | | 282: 255-264. |
| 23S | 2052 | “A to C” | Prevents ErmE | E. coli | Vester B, Nielsen AK, Hansen LH, Douthwaite S. |
| | | | methylation. c | | 1998. ErmE Methyltransferase Recognition |
| | | | | | Elements in RNA Substrates. J. Mol. Biol. |
| | | | | | 282: 255-264. |
| 23S | 2052 | “A to G” | Like A2052C. c | E. coli | Vester B, Nielsen AK, Hansen LH, Douthwaite S. |
| | | | | | 1998. ErmE Methyltransferase Recognition |
| | | | | | Elements in RNA Substrates. J. Mol. Biol. |
| | | | | | 282: 255-264. |
| 23S | 2052 | “A to U” | Like A2052C. c | E. coli | Vester B, Nielsen AK, Hansen LH, Douthwaite S. |
| | | | | | 1998. ErmE Methyltransferase Recognition |
| | | | | | Elements in RNA Substrates. J. Mol. Biol. |
| | | | | | 282: 255-264. |
| 23S | 2057 | “G to A” | Eryr, Clinidamycin | E. coli | 1. Ettayebi, M., Prasad, S. M., and Morgan, E. A. |
| | | | (Cd)s, | | (1985) J. Bacteriol. 162, 551-557 2. |
| | | | Chloramphemicol | | Aaagard, C. and Douthwaite, S. (1994) Proc. |
| | | | (Cm)r; reduces | | Natl. Acad. Sci. USA 91, 2989-2993. 3. |
| | | | methylation of 23S | | Vester, B., Hansen, L. H., and Douthwaite, S. |
| | | | rRNA by ErmE. | | (1995) RNA 1, 501-509. |
| 23S | 2057 | “G to A” | Eyrr. | Chlamydomonas | Harris, E. H., Burkhart, B. D., Gilham, N. W., |
| | | | | reinhardtii | and Boynton, J. E. (1989) Genetics 123, 281-292. |
| 23S | 2057 | “G to A” | Slightly Eryr; | E. coli | Vester, B., Hansen, L. H., and Douthwaite, S. |
| | | | reduced | | (1995) RNA 1, 501-509. |
| | | | methylation. | | |
| | | | Double mutation | | |
| | | | (G2057A/C2661U) | | |
| 23S | 2057 | “G to A” | Eryr, Cdr, Cmr. | E. coli | 1. Douthwaite, S. (1992) J. Bacteriol. 174, |
| | | | Double mutation | | 1333-1338. 2. Aaagard, C. and Douthwaite, S. |
| | | | (G2057A/G2032A) | | (1994) Proc. Natl. Acad. Sci. USA 91, |
| | | | | | 2989-2993. 3. Vester B., Hansen, L. H., and |
| | | | | | Douthwaite, S. (1995) RNA 1, 501-509. |
| 23S | 2057 | G to A | Ery-R, Lin-S | Chlamydomonas | Harris, E. H., B. D. Burkhart, N. W. Gillham, |
| | | | | reinhardtii chl. | and J. E. Boynton. 1989. Antibiotic |
| | | | | | resistance mutations in the chloroplast 16S |
| | | | | | and 23S rRNA genes of Chlamydomonas |
| | | | | | reinhardtii: correlation of genetic and |
| | | | | | physical maps of the chloroplast genome. |
| | | | | | Genetics. |
| 23S | 2057 | G to A | Ery-R, M16-S, Lin- | Escherichia coli | Ettayebi, M., S. M. Prasad, and E. A. Morgan. |
| | | | S, SB-S | | 1985. Chloramphenicol- |
| | | | | | erythromycin resistance mutations in a 23S |
| | | | | | rRNA gene of Escherichia coli. J. Bacteriol. |
| | | | | | 162: 551-557. |
| 23S | 2057 | G to A | Ery-LR, M16-S | Propionibacteria | Ross, J. I., E. A. Eady, J. H. Cove, C. E. Jones, |
| | | | | | A. H. Ratyal, Y. W. Miller, S. Vyakrnam, |
| | | | | | and W. J. Cunliffe. 1997. |
| | | | | | Clinical resistance to erythromycin and |
| | | | | | clindamycin in cutaneous propionibacteria |
| | | | | | isolated from acne patients is associated |
| | | | | | with mutatio |
| 23S | 2057 | GG to AA | Ery-R, Lin-R | Escherichia coli | Douthwaite, S. 1992. Functional |
| | | | | | interactions within 23S rRNA involving the |
| | | | | | peptidyltransferase center. J. Bacteriol. |
| | | | | | 174: 1333-1338. |
| 23S | 2058 | “A to G” | Eryr, Lincomycin | Chlamydomonas | Harris, E. H., Burkhart, B. D., Gilham, N. W., |
| | | | and clindamycin | reinhardtii | and Boynton, J. E. (1989) Genetics 123, |
| | | | resistance. | | 281-292. |
| 23S | 2058 | “A to G” | Clarithromycin | Helecobacter | Versalovic, J., Shortridge, D., Kibler, K., |
| | | | resistance | pylori | Griffy, M. V., Beyer, J., Flamm, R. K., |
| | | | | | Tenaka, S. K., Graham, D. Y., and Go, M. F. |
| | | | | | (1996) Antimicrob. Agents Chemother. 40, |
| | | | | | 477-480. |
| 23S | 2058 | “A to G” | Eryr, Cdr, Cms; | E. coli | 1. Vester, B. and Garrett, R. A. (1988) |
| | | | abolishes | | EMBO J. 7, 3577-3587. 2. Aaagard, C. and |
| | | | methylation of 23S | | Douthwaite, S. (1994) Proc. Natl. Acad. Sci. |
| | | | rRNA by ErmE. | | USA 91, 2989-2993. 3. Vester, B., Hansen, L. H., |
| | | | | | and Douthwaite, S. (1995) RNA 1, |
| | | | | | 501-509. |
| 23S | 2058 | “A to G” | Erythromycin | Yeast | Sor, F. and Fukuhara, H. (1982) Nucleic |
| | | | resistance. | mitochondria | Acids Res. 10, 6571-6577. |
| 23S | 2058 | “A to G” | Lincomycin | Solanum nigrum | Kavanagh, T. A., O'Driscoll, K. M., McCabe, P. F., |
| | | | resistance. | | and Dix, P. J. (1994) Mol. Gen. Genet. |
| | | | | | 242, 675-680. |
| 23S | 2058 | “A to G” | Lincomycin | Tobacco | Cseplö, A., Etzold, T., Schell, J., and |
| | | | resistance. | chloroplasts | Schreier, P. H. (1988) Mol. Genet. 214, 295-299. |
| 23S | 2058 | “A to G” | EryS, Cds, Cms. | E. coli | 1. Douthwaite, S. (1992) J. Bacteriol. 174, |
| | | | Double mutation | | 1333-1338. 2. Aaagard, C. and Douthwaite, S. |
| | | | (A2058G/G2032A) | | (1994) Proc. Natl. Acad. Sci. USA 91, |
| | | | | | 2989-2993. 3. Vester, B., Hansen, L. H., |
| | | | | | and Douthwaite, S. (1995) RNA 1, 501-509. |
| 23S | 2058 | “A to U” | Eryhs, Cds, Cms. | E. coli | 1. Douthwaite, S. (1992) J. Bacteriol. 174, |
| | | | Double mutation | | 1333-1338. 2. Aaagard, C. and Douthwaite, S. |
| | | | (A2058U/G2032A) | | (1994) Proc. Natl. Acad. Sci. USA 91, |
| | | | | | 2989-2993. 3. Vester, B., Hansen, L. H., |
| | | | | | and Douthwaite, S. (1995) RNA 1, 501-509. |
| 23S | 2058 | “A to C” | Confers | E. coli | Hansen LH, Mauvais P, Douthwaite S. |
| | | | resistance to the | | 1999. The macrolide-kelotide antibiotic |
| | | | MLS drugs and | | binding site is formed by structures in |
| | | | chloramphenicol. | | domains II and V of 23S ribosomal RNA. |
| | | | | | Molecular Microbiology 31 (2): 623-631. |
| 23S | 2058 | “A to G” | Like A2058C | E. coli | Hansen LH, Mauvais P, Douthwaite S. |
| | | | | | 1999. The macrolide-kelotide antibiotic |
| | | | | | binding site is formed by structures in |
| | | | | | domains II and V of 23S ribosomal RNA. |
| | | | | | Molecular Microbiology 31 (2): 623-631. |
| 23S | 2058 | “A to U” | Like A2058C | E. coli | Hansen LH, Mauvais P, Douthwaite S. |
| | | | | | 1999. The macrolide-kelotide antibiotic |
| | | | | | binding site is formed by structures in |
| | | | | | domains II and V of 23S ribosomal RNA. |
| | | | | | Molecular Microbiology 31 (2): 623-631. |
| 23S | 2058 | A to G/U | Ery-R, Tyl-R, Lin-R | Brachyspira | Karlsson, M., C. Fellstrom, M. U. Heldtander, |
| | | | | hyodysenteriae | K. E. Johansson, and A. Franklin. |
| | | | | | 1999. Genetic basis of macrolide |
| | | | | | and lincosamide resistance in Brachyspira |
| | | | | | (Serpulina) hyodysenteriae. FEMS |
| | | | | | Microbiol. Lett. 172: 255-260. |
| 23S | 2058 | A to G | Ery-R, Lin-R | Chlamydomonas | Harris, E. H., B. D. Burkhart, N. W. Gillham, |
| | | | | reinhardtii chl. | and J. E. Boynton. 1989. Antibiotic |
| | | | | | resistance mutations in the chloroplast 16S |
| | | | | | and 23S rRNA genes of Chlamydomonas |
| | | | | | reinhardtii: correlation of genetic and |
| | | | | | physical maps of the chloroplast genome. |
| | | | | | Genetics. |
| 23S | 2058 | A to G | Ery-R, Lin-R | Escherichia coli | Douthwaite, S. 1992. Functional |
| | | | | | interactions within 23S rRNA involving the |
| | | | | | peptidyltransferase center. J. Bacteriol. |
| | | | | | 174: 1333-1338. Vester, B., and R. A. Garrett. |
| | | | | | 1987. A plasmid-coded and site- |
| | | | | | directed mutation in Escherichia coli 23S |
| | | | | | RNA that confers |
| 23S | 2058 | A to U | MLSB-R | Escherichia coli | Sigmund, C. D., M. Ettayebi, and E. A. Morgan. |
| | | | | | 1984. Antibiotic resistance |
| | | | | | mutations in 16S and 23S ribosomal RNA |
| | | | | | genes of Escherichia coli. Nucl Acids Res. |
| | | | | | 12: 4653-4663. |
| 23S | 2058 | A to C | Clr-R | Helicobacter | Stone, G. G., D. Shortridge, R. K. Flamm, J. Versalovic, |
| | | | | pylori | J. Beyer, K. Idler, L. Zulawinski, |
| | | | | | and S. K. Tanaka. 1996. Identification of a |
| | | | | | 23S rRNA gene mutation in clarithromycin- |
| | | | | | resistant Helicobacter pylori. Helicobacter. |
| | | | | | 1: 227-228. |
| 23S | 2058 | A to C | Mac-R, Lin-R | Helicobacter | Occhialini, A., M. Urdaci, F. Doucet- |
| | | | | pylori | Populaire, C. M. Bébéar, H. Lamouliatte, |
| | | | | | and F. Mégraud. 1997. Macrolide |
| | | | | | resistance in Helicobacter pylori: rapid |
| | | | | | detection of point mutations and assays of |
| | | | | | macrolide binding to ribosomes. Antimicrob. |
| | | | | | Agents Chemothe |
| 23S | 2058 | A to C | MLSB-R | Helicobacter | Wang, G., and D. E. Taylor. 1998. Site- |
| | | | | pylori | specific mutations in the 23S rRNA gene of |
| | | | | | Helicobacter pylori confer two types of |
| | | | | | resistance to macrolide-lincosamide- |
| | | | | | streptogramin B antibiotics. Antimicrob. |
| | | | | | Agents Chemother. 42: 1952-1958. |
| 23S | 2058 | A to C | Cla-R | Helicobacter | Debets-Ossenkopp, Y. J., A. B. Brinkman, |
| | | | | pylori | E. J. Kuipers, C. M. Vandenbroucke- |
| | | | | | Grauls, and J. G. Kusters. 1998. Explaining |
| | | | | | the bias in the 23S rRNA gene mutations |
| | | | | | associated with clarithromycin resistance in |
| | | | | | clinical isolates of Helicobacter pylori. |
| | | | | | Antimi |
| 23S | 2058 | A to G | Cla-R | Helicobacter | Versalovic, J., D. Shortridge, K. Kibler, M. V. Griffy, |
| | | | | pylori | J. Beyer, R. K. Flamm, S. K. Tanaka, |
| | | | | | D. Y. Graham, and M. F. Go. 1996. |
| | | | | | Mutations in 23S rRNA are associated with |
| | | | | | clarithromycin resistance in Helicobacter |
| | | | | | pylori. Antimicrob. Agents Chemother. 40: 4 |
| 23S | 2058 | A to G | Mac-R, Lin-R | Helicobacter | Occhialini, A., M. Urdaci, F. Doucet- |
| | | | | pylori | Populaire, C. M. Bébéar, H. Lamouliatte, |
| | | | | | and F. Mégraud. 1997. Macrolide |
| | | | | | resistance in Helicobacter pylori: rapid |
| | | | | | detection of point mutations and assays of |
| | | | | | macrolide binding to ribosomes. Antimicrob. |
| | | | | | Agents Chemothe |
| 23S | 2058 | A to G | MLSB-R | Helicobacter | Wang, G., and D. E. Taylor. 1998. Site- |
| | | | | pylori | specific mutations in the 23S rRNA gene of |
| | | | | | Helicobacter pylori confer two types of |
| | | | | | resistance to macrolide-lincosamide- |
| | | | | | streptogramin B antibiotics. Antimicrob. |
| | | | | | Agents Chemother. 42: 1952-1958. |
| 23S | 2058 | A to G | Cla-R | Helicobacter | Debets-Ossenkopp, Y. J., A. B. Brinkman, |
| | | | | pylori | E. J. Kuipers, C. M. Vandenbroucke- |
| | | | | | Grauls, and J. G. Kusters. 1998. Explaining |
| | | | | | the bias in the 23S rRNA gene mutations |
| | | | | | associated with clarithromycin resistance in |
| | | | | | clinical isolates of Helicobacter pylori. |
| | | | | | Antimi |
| 23S | 2058 | A to U | MLSB-R | Helicobacter | Wang, G., and D. E. Taylor. 1998. Site- |
| | | | | pylori | specific mutations in the 23S rRNA gene of |
| | | | | | Helicobacter pylori confer two types of |
| | | | | | resistance to macrolide-lincosamide- |
| | | | | | streptogramin B antibiotics. Antimicrob. |
| | | | | | Agents Chemother. 42: 1952-1958. |
| 23S | 2058 | A to U | Cla-R | Helicobacter | Debets-Ossenkopp, Y. J., A. B. Brinkman, |
| | | | | pylori | E. J. Kuipers, C. M. Vandenbroucke- |
| | | | | | Grauls, and J. G. Kusters. 1998. Explaining |
| | | | | | the bias in the 23S rRNA gene mutations |
| | | | | | associated with clarithromycin resistance in |
| | | | | | clinical isolates of Helicobacter pylori. |
| | | | | | Antimi |
| 23S | 2058 | A to G | Clr-R | Mycobacterium | Wallace, R. J., Jr., A. Meier, B. A. Brown, Y. Zhang, |
| | | | | abscessus | P. Sander, G. O. Onyi, and E. C. Böttger. |
| | | | | | 1996. Genetic basis for |
| | | | | | clarithromycin resistance among isolates of |
| | | | | | Mycobacterium chelonae and |
| | | | | | Mycobacterium abscessus. Antimicrob. |
| | | | | | Agents Chemother. 40: 1676 |
| 23S | 2058 | A to C/G/U | Clr-R | Mycobacterium | Nash, K. A., and C. B. Inderlied. 1995. |
| | | | | avium | Genetic basis of macrolide resistance in |
| | | | | | Mycobacterium avium isolated from patients |
| | | | | | with disseminated disease. Antimicrob. |
| | | | | | Agents Chemother. 39: 2625-2630. |
| 23S | 2058 | A to C/G/U | Clr-R | Mycobacterium | Nash, K. A., and C. B. Inderlied. 1995. |
| | | | | avium | Genetic basis of macrolide resistance in |
| | | | | | Mycobacterium avium isolated from patients |
| | | | | | with disseminated disease. Antimicorb. |
| | | | | | Agents Chemother. 39: 2625-2630. |
| 23S | 2058 | A to C/G | Clr-R | Mycobacterium | Wallace, R. J., Jr., A. Meier, B. A. Brown, Y. Zhang, |
| | | | | chelonae | P. Sander, G. O. Onyi, and E. C. Böttger. |
| | | | | | 1996. Genetic basis for |
| | | | | | clarithromycin resistance among isolates of |
| | | | | | Mycobacterium chelonae and |
| | | | | | Mycobacterium abscessus. Antimicrob. |
| | | | | | Agents Chemother. 40: 1676 |
| 23S | 2058 | A to C/G/U | Clr-R | Mycobacterium | Meier, A., P. Kirschner, B. Springer, V. A. Steingrube, |
| | | | | intracellulare | B. A. Brown, R. J. Wallace, Jr., |
| | | | | | and E. C. Böttger. 1994. Identification of |
| | | | | | mutations in 23S rRNA gene of |
| | | | | | clarithromycin-resistant Mycobacterium |
| | | | | | intracellulare. Antimicrob. Agents |
| | | | | | Chemother. 38: 38 |
| 23S | 2058 | A to U | Clr-R | Mycobacterium | Burman, W. J., B. L. Stone, B. A. Brown, R. J. Wallace, |
| | | | | kansasii | Jr., and E. C. Böttger. 1998. |
| | | | | | AIDS-related Mycobacterium kansasii |
| | | | | | infection with initial resistance to |
| | | | | | clarithromycin. Diagn. Microbiol. Infect. Dis. |
| | | | | | 31: 369-371. |
| 23S | 2058 | A to G | Clr-R | Mycobacterium | Sander, P., T. Prammananan, A. Meier, K. Frischkorn, |
| | | | | smegmatis | and E. C. Böttger. 1997. The |
| | | | | | role of ribosomal RNAs in macrolide |
| | | | | | resistance. Mol. Microbiol. 26: 469-480. |
| 23S | 2058 | A to G | Ery-HR, Spi-MR, | Mycoplasma | Lucier, T. S., K. Heitzman, S. K. Liu, and P. C. Hu. |
| | | | Tyl-S, Lin-HR | pneumoniae | 1995. Transition mutations in the 23S |
| | | | | | rRNA of erythromycin-resistant isolates of |
| | | | | | Mycoplasma pneumoniae. Antimicrob. |
| | | | | | Agents Chemother. 39: 2770-2773. |
| 23S | 2058 | A to G | MLSB-R | Propionibacteria | Ross, J. I., E. A. Eady, J. H. Cove, C. E. Jones, |
| | | | | | A. H. Ratyal, Y. W. Miller, S. Vyakrnam, |
| | | | | | and W. J. Cunliffe. 1997. Clinical |
| | | | | | resistance to erythromycin and clindamycin |
| | | | | | in cutaneous propionibacteria isolated from |
| | | | | | acne patients is associated with mutatio |
| 23S | 2058 | A to G | MLSB-R | Streptococcus | Tait-Kamradt, A., T. Davies, M. Cronan, M. R. Jacobs, |
| | | | | pneumoniae | P. C. Appelbaum, and J. Sutcliffe. |
| | | | | | 2000. Mutations in 23S rRNA and |
| | | | | | L4 ribosomal protein account for resistance |
| | | | | | in Pneumococcal strains selected in vitro by |
| | | | | | macrolide passage. Antimicrobial Agents |
| | | | | | and |
| 23S | 2058 | A to G | MLSB-R | Streptomyces | Pernodet, J. L., F. Boccard, M. T. Alegre, M. H. Blondelet- |
| | | | | ambofaciens | Rouault, and M. Guerineau. |
| | | | | | 1988. Resistance to macrolides, |
| | | | | | lincosamides and streptogramin type B |
| | | | | | antibiotics due to a mutation in an rRNA |
| | | | | | operon of Streptomyces ambofaciens. |
| | | | | | EMBO J. 7: 277-282. |
| 23S | 2058 | A to G | Ery-R | Saccharomyces | Sor, F., and H. Fukuhara. 1982. |
| | | | | cerevisiae mit. | Identification of two erythromycin resistance |
| | | | | | mutations in the mitochondrial gene coding |
| | | | | | for the large ribosomal RNA in yeast. |
| | | | | | Nucleic Acids Res. 10: 6571-6577. |
| 23S | 2058 | A to G | Ery-R | Treponema | Stamm, L. V., and H. L. Bergen. 2000. A |
| | | | | pallidum | point mutation associated with bacterial |
| | | | | | macrolide resistance is present in both 23S |
| | | | | | rRNA genes of an erythromycin-resistant |
| | | | | | Treponema pallidum clinical isolate [letter]. |
| | | | | | Antimicrob Agents Chemother. 44: 806-807. |
| 23S | 2059 | “A to G” | Clarithomycin | Helecobacter | Versalovic, J., Shortridge, D., Kibler, K., |
| | | | resistance. | pylori | Griffy, M. V., Beyer, J., Flamm, R. K., Tanaka, S. K., |
| | | | | | Graham, D. Y., and Go, M. F. (1996) |
| | | | | | Antimicrob. Agents and Chemother. 40, 477-480. |
| 23S | 2059 | “A to G” | Lincomycin | Tobacco | Cseplö, A., Etzold, T., Schell, J., and |
| | | | resistance. | chloroplasts | Schreier, P. H. (1988) Mol. Genet. 214, 295-299. |
| 23S | 2059 | “A to C” | Conferred | E. coli | Hansen LH, Mauvais P, Douthwaite S. 1999. |
| | | | resistance to the | | The macrolide-kelotide antibiotic binding site |
| | | | MLS drugs and | | is formed by structures in domains II and V |
| | | | chloramphenicol. | | of 23S ribosomal RNA. Molecular |
| | | | | | Microbiology 31 (2): 623-631. |
| 23S | 2059 | “A to G” | Like A2059C | E. coli | Hansen LH, Mauvais P, Douthwaite S. 1999. |
| | | | | | The macrolide-kelotide antibiotic binding site |
| | | | | | is formed by structures in domains II and V |
| | | | | | of 23S ribosomal RNA. Molecular |
| | | | | | Microbiology 31 (2): 623-631. |
| 23S | 2059 | “A to U” | Like A2059C | E. coli | Hansen LH, Mauvais P, Douthwaite S. 1999. |
| | | | | | The macrolide-kelotide antibiotic binding site |
| | | | | | is formed by structures in domains II and V |
| | | | | | of 23S ribosomal RNA. Molecular |
| | | | | | Microbiology 31 (2): 623-631. |
| 23S | 2059 | A to C | Mac-R, Lin-R, SB-S | Helicobacter | Wang, G., and D. E. Taylor. 1998. Site- |
| | | | | pylori | specific mutations in the 23S rRNA gene of |
| | | | | | Helicobacter pylori confer two types of |
| | | | | | resistance to macrolide-lincosamide- |
| | | | | | streptogramin B antibiotics. Antimicrob. |
| | | | | | Agents Chemother. 42: 1952-1958. |
| 23S | 2059 | A to C | Clr-R | Helicobacter | Debets-Ossenkopp, Y. J., A. B. Brinkman, E. J. Kuipers, |
| | | | | pylori | C. M. Vandenbroucke-Grauls, |
| | | | | | and J. G. Kusters. 1998. Explaining the bias |
| | | | | | in the 23S rRNA gene mutations associated |
| | | | | | with clarithromycin resistance in clinical |
| | | | | | isolates of Helicobacter pylori. Antimi |
| 23S | 2059 | A to G | Clr-R | Helicobacter | Versalovic, J., D. Shortridge, K. Kibler, M. V. Griffy, |
| | | | | pylori | J. Beyer, R. K. Flamm, S. K. Tanaka, |
| | | | | | D. Y. Graham, and M. F. Go. 1996. |
| | | | | | Mutations in 23S rRNA are associated with |
| | | | | | clarithromycin resistance in Helicobacter |
| | | | | | pylori. Antimicrob. Agents Chemother. 40: 4 |
| 23S | 2059 | A to G | Mac-R, Lin-R | Helicobacter | Occhialini, A., M. Urdaci, F. Doucet- |
| | | | | pylori | Populaire, C. M. Bébéar, H. Lamouliatte, |
| | | | | | and F. Mégraud. 1997. Macrolide resistance |
| | | | | | in Helicobacter pylori: rapid detection of |
| | | | | | point mutations and assays of macrolide |
| | | | | | binding to ribosomes. Antimicrob. Agents |
| | | | | | Chemothe |
| 23S | 2059 | A to G | Mac-R, Lin-R, | Helicobacter | Wang, G., and D. E. Taylor. 1998. Site- |
| | | | SB-S | pylori | specific mutations in the 23S rRNA gene of |
| | | | | | Helicobacter pylori confer two types of |
| | | | | | resistance to macrolide-lincosamide- |
| | | | | | streptogramin B antibiotics. Antimicrob. |
| | | | | | Agents Chemother. 42: 1952-1958. |
| 23S | 2059 | A to G | Cla-R | Helicobacter | Debets-Ossenkopp, Y. J., A. B. Brinkman, E. J. Kuipers, |
| | | | | pylori | C. M. Vandenbroucke-Grauls, |
| | | | | | and J. G. Kusters. 1998. Explaining the bias |
| | | | | | in the 23S rRNA gene mutations associated |
| | | | | | with clarithromycin resistance in clinical |
| | | | | | isolates of Helicobacter pylori. Antimi |
| 23S | 2059 | A to C/G | Clr-R | Mycobacterium | Wallace, R. J., Jr., A. Meier, B. A. Brown, Y. Zhang, |
| | | | | abscessus | P. Sander, G. O. Onyi, and E. C. Böttger. |
| | | | | | 1996. Genetic basis for |
| | | | | | clarithromycin resistance among isolates of |
| | | | | | Mycobacterium chelonae and |
| | | | | | Mycobacterium abscessus. Antimicrob. |
| | | | | | Agents Chemother. 40: 1676 |
| 23S | 2059 | A to G | Clr-R | Mycobacterium | Wallace, R. J., Jr., A. Meier, B. A. Brown, Y. Zhang, |
| | | | | chelonae | P. Sander, G. O. Onyi, and E. C. Böttger. |
| | | | | | 1996. Genetic basis for |
| | | | | | clarithromycin resistance among isolates of |
| | | | | | Mycobacterium chelonae and |
| | | | | | Mycobacterium abscessus. Antimicrob. |
| | | | | | Agents Chemother. 40: 1676 |
| 23S | 2059 | A to C | Clr/Azm-R | Mycobacterium | Meier, A., P. Kirschner, B. Springer, V. A. Steingrube, |
| | | | | intracellulare | B. A. Brown, R. J. Wallace, Jr., |
| | | | | | and E. C. Böttger. 1994. Identification of |
| | | | | | mutations in 23S rRNA gene of |
| | | | | | clarithromycin-resistant Mycobacterium |
| | | | | | intracellulare. Antimicrob. Agents |
| | | | | | Chemother. 38: 38 |
| 23S | 2059 | A to C | Clr/Azm-R | Mycobacterium | Meier, A., P. Kirschner, B. Springer, V. A. Steingrube, |
| | | | | avium | B. A. Brown, R. J. Wallace, Jr., |
| | | | | | and E. C. Böttger. 1994. Identification of |
| | | | | | mutations in 23S rRNA gene of |
| | | | | | clarithromycin-resistant Mycobacterium |
| | | | | | intracellulare. Antimicrob. Agents |
| | | | | | Chemother. 38: 38 |
| 23S | 2059 | A to G | Clr-R | Mycobacterium | Sander, P., T. Prammananan, A. Meier, K. Frischkorn, |
| | | | | smegmatis | and E. C. Böttger. 1997. The |
| | | | | | role of ribosomal RNAs in macrolide |
| | | | | | resistance. Mol. Microbiol. 26: 469-480. |
| 23S | 2059 | A to G | Ery-MR, Spi-HR, | Mycoplasma | Lucier, T. S., K. Heitzman, S. K. Liu, and P. C. Hu. |
| | | | Tyl-LR, Lin-MR | pneumoniae | 1995. Transition mutations in the 23S |
| | | | | | rRNA of erythromycin-resistant isolates of |
| | | | | | Mycoplasma pneumoniae. Antimicrob. |
| | | | | | Agents Chemother. 39: 2770-2773. |
| 23S | 2059 | A to G | Mac-R | Streptococcus | Tait-Kamradt, A., T. Davies, M. Cronan, M. R. Jacobs, |
| | | | | pneumoniae | P. C. Appelbaum, and J. Sutcliffe. |
| | | | | | 2000. Mutations in 23S rRNA and |
| | | | | | L4 ribosomal protein account for resistance |
| | | | | | in Pneumococcal strains selected in vitro by |
| | | | | | macrolide passage. Antimicrobial Agents |
| | | | | | and |
| 23S | 2059 | A to G | Mac-HR, Lin-LR | Propionibacteria | Ross, J. I., E. A. Eady, J. H. Cove, C. E. Jones, |
| | | | | | A. H. Ratyal, Y. W. Miller, S. Vyakrnam, |
| | | | | | and W. J. Cunliffe. 1997. Clinical |
| | | | | | resistance to erythromycin and clindamycin |
| | | | | | in cutaneous propionibacteria isolated from |
| | | | | | acne patients is associated with mutatio |
| 23S | 2060 | “A to C” | | E. coli | Vester, B. and Garrett, R. A. (1988) EMBO |
| | | | | | J. 7, 3577-3587. |
| 23S | 2061 | “G to A” | Chloramphenicol | Rat mitochondria | Vester, B. and Garrett, R. A. (1988) EMBO |
| | | | resistance | | J. 7, 3577-3587. |
| 23S | 2062 | “A to C” | Chloramphenicol | Halobacterium | Mankin, A. S. and Garrett, R. A. (1991) J. |
| | | | resistance. | halobium | Bacteriol. 173, 3559-3563. |
| 23S | 2251 | “G to A” | Dominant lethal; | E. coli | Green, R., Samaha, R., and Noller, H. |
| | | | Abolished both | | (1997). J. Mol. Biol. 266, 40-50. |
| | | | binding of tRNA | | |
| | | | and peptidyl | | |
| | | | transferase activity. | | |
| 23S | 2251 | “G to A” | Dominant lethal; | E. coli | Gregory, S. T. and Dahlberg, A. E. |
| | | | subunit association | | (unpublished). |
| | | | defect. | | |
| 23S | 2251 | “G to C” | Dominant lethal | E. coli | Gregory, S. T. and Dahlberg, A. E. |
| | | | subunit association | | (unpublished). |
| | | | defect. | | |
| 23S | 2251 | “G to U” | Dominant lethal | E. coli | Gregory, S. T. and Dahlberg, A. E. |
| | | | subunit association | | (unpublished). |
| | | | defect. | | |
| 23S | 2251 | “G to U” | Dominant lethal; | E. coli | Green, R., Samaha, R., and Noller, H. |
| | | | Abolished both | | (1997). J. Mol. Biol. 266, 40-50. |
| | | | binding of tRNA | | |
| | | | and peptidyl | | |
| | | | transferase activity. | | |
| 23S | 2251 | “G to A” | Dominant lethal; | E. coli | Gregory ST, Dahlberg AE, 1999. Mutations |
| | | | impairs peptidyl | | in the Conserved P Loop Perturb the |
| | | | transferase activity; | | Conformation of Two Structural Elements |
| | | | induces DMS | | in the Peptidyl Transferase Center of 23 S |
| | | | reactivity; induces | | Ribosomal RNA. J. Mol. Biol. 285: 1475-1483. |
| | | | kethoxal reactivity | | |
| | | | in G2238, G2409, | | |
| | | | G2410, G2529, | | |
| | | | and G2532; | | |
| | | | enhances CMCT | | |
| | | | reactivity in G2238; | | |
| | | | induces kethoxal | | |
| | | | and CMCT | | |
| | | | reactivity in G2269 | | |
| | | | and G2271; | | |
| | | | induces CMCT | | |
| | | | reactivity in U2272 | | |
| | | | and U2408; | | |
| | | | enhances kethoxal | | |
| | | | reactivity in G2253. | | |
| 23S | 2252 | “G to A” | Less than 5% of | E. coli | Porse, B. T., Thi-Ngoc, H. P., and Garrett, R. A. |
| | | | control level | | (1996) J. Mol. Biol. 264: 472-486. |
| | | | peptidyl transferase | | |
| | | | activity. | | |
| 23S | 2252 | “G to C” | Less than 5% of | E. coli | Porse, B. T., Thi-Ngoc, H. P., and Garrett, R. A. |
| | | | control level | | (1996) J. Mol. Biol. 264: 472-486. |
| | | | peptidyl transferase | | |
| | | | activity. | | |
| 23S | 2252 | “G to U” | Less than 5% of | E. coli | Porse, B. T., Thi-Ngoc, H. P., and Garrett, R. A. |
| | | | control level | | (1996) J. Mol. Biol. 264: 472-486. |
| | | | peptidyl transferase | | |
| | | | activity. | | |
| 23S | 2252 | “G to C” | Reduced rate of | E. coli | 1. Lieberman, K. R. and Dahlberg, A. E. |
| | | | peptidyl transferase | | (1994) J. Biol. Chem. 269, 16163-16169. |
| | | | bond formation in | | 2. Samaha, R. R., Green R., and Noller, H. F. |
| | | | vitro; severely | | (1995) Nature 377, 309-314. 3. |
| | | | detrimental to cell | | O'Connor, M., Brunelli, C. A., Firpo, M. A., |
| | | | growth. Double | | Gregory, S. T., Lieberman, K. R., Lodmell, J. S., |
| | | | mutation | | Moine, H., Van Ryk, D. I., and |
| | | | (G2252C/G2253C). | | Dahlberg, A. E. (1995) Cell Biol. 73, 859-868. |
| 23S | 2252 | “G to A” | Severely detrimental | E. coli | 1. Gregory, S. T., Lieberman, K. R., and |
| | | | to cell growth; | | Dahlberg, A. E. (1994) Nucleic Acids Res. |
| | | | promoted | | 22, 279-284. 2. Lieberman, K. R. and |
| | | | frameshifting and | | Dahlberg, A. E. (1994) J. Biol. Chem. 269, |
| | | | readthrough of | | 16163-16169. |
| | | | nonsense codons. | | |
| 23S | 2252 | “G to C” | Severely detrimental | E. coli | 1. Gregory, S. T., Lieberman, K. R., and |
| | | | to cell growth; | | Dahlberg, A. E. (1994) Nucleic Acids Res. |
| | | | promoted | | 22, 279-284. 2. Lieberman, K. R. and |
| | | | frameshifting and | | Dahlberg, A. E. (1994) J. Biol. Chem. 269, |
| | | | readthrough of | | 16163-16169. |
| | | | nonsense codons. | | |
| 23S | 2252 | “G to U” | Severely detrimental | E. coli | 1. Gregory, S. T., Lieberman, K. R., and |
| | | | to cell growth; | | Dahlberg, A. E. (1994) Nucleic Acids Res. |
| | | | promoted | | 22, 279-284. 2. Lieberman, K. R. and |
| | | | frameshifting and | | Dahlberg, A. E. (1994) J. Biol. Chem. 269, |
| | | | readthrough of | | 16163-16169. |
| | | | nonsense codons. | | |
| 23S | 2252 | “G to A” | Dominant lethal; | E. coli | Gregory ST, Dahlberg AE, 1999. |
| | | | impairs peptidyl | | Mutations in the Conserved P Loop |
| | | | transferase activity; | | Perturb the Conformation of Two |
| | | | induces DMS | | Structural Elements in the Peptidyl |
| | | | reactivity; induces | | Transferase Center of 23 S Ribosomal |
| | | | kethoxal reactivity in | | RNA. J. Mol. Biol. 285: 1475-1483. |
| | | | G2238, G2409, | | |
| | | | G2410, G2529, and | | |
| | | | G2532; enhances | | |
| | | | CMCT reactivity in | | |
| | | | G2238; induces | | |
| | | | kethoxal and CMCT | | |
| | | | reactivity in G2269 | | |
| | | | and G2271; induces | | |
| | | | CMCT reactivity in | | |
| | | | U2272 and U2408; | | |
| | | | enhances kethoxal | | |
| | | | reactivity in G2253. | | |
| 23S | 2252 | “G to A” | Interfere with the | E. coli | Bocchetta M, Xiong L, Mankin AS. 1998. |
| | | | building of peptidyl- | | 23S rRNA positions essential for tRNA |
| | | | tRNA to P site of 50S | | binding in ribosomal functional sites. Proc. |
| | | | subunit. | | Natl. Acad. Sci. 95: 3525-3530. |
| 23S | 2252 | “G to C” | Interferes with the | E. coli | Bocchetta M, Xiong L, Mankin AS. 1998. |
| | | | binding of peptidyl- | | 23S rRNA positions essential for tRNA |
| | | | tRNA to P site of 50S | | binding in ribosomal functional sites. Proc. |
| | | | subunit | | Natl. Acad. Sci. 95: 3525-3530. |
| 23S | 2252 | “G to U” | Interferes with the | E. coli | Bocchetta M, Xiong L, Mankin AS. 1998. |
| | | | binding of peptidyl- | | 23S rRNA positions essential for tRNA |
| | | | tRNA to P site of 50S | | binding in ribosomal functional sites. Proc. |
| | | | subunit | | Natl. Acad. Sci. 95: 3525-3530. |
| 23S | 2252 | “G to U” | Dominant lethal; | E. coli | Nitta I, Ueda T, Watanabe K. 1998. |
| | | | suppressed AcPhe- | | Possible involvement of Escherichia coli |
| | | | Phe formation; | | 23S ribosomal RNA in peptide bond |
| | | | suppressed peptide | | formation. RNA 4: 257-267. |
| | | | bond formation. c | | |
| 23S | 2253 | “G to C” | 42% control level | E. coli | Porse, B. T., Thi-Ngoc, H. P., and Garrett, R. A. |
| | | | peptidyl transferase | | (1996) J. Mol. Biol. 264: 472-486. |
| | | | activity. | | |
| 23S | 2253 | “G to C” | Slow growth rate. | E. coli | Gregory, S. T., Lieberman, K. R., and |
| | | | | | Dahlberg, A. E. (1994) Nucleic Acids Res. |
| | | | | | 22, 279-284. |
| 23S | 2253 | “G to C” | Promoted | E. coli | 1. Lieberman, K. R. and Dahlberg, A. E. |
| | | | frameshifting and | | (1994) J. Biol. Chem. 269, 16163-16169. |
| | | | readthrough of | | 2. Samaha, R. R., Green R., and Noller, H. F. |
| | | | nonsense codons. | | (1995) Nature 377, 309-314. 3. |
| | | | | | O'Connor, M., Brunelli, C. A., Firpo, M. A., |
| | | | | | Gregory, S. T., Lieberman, K. R., Lodmell, J. S., |
| | | | | | Moine, H., Van Ryk, D. I., and |
| | | | | | Dahlberg, A. E. (1995) Cell Biol. 73, 859-868. |
| 23S | 2253 | “G to A” | Promoted | E. coli | 1. Lieberman, K. R. and Dahlberg, A. E. |
| | | | frameshifting and | | (1994) J. Biol. Chem. 269, 16163-16169. |
| | | | readthrough of | | 2. Samaha, R. R., Green R., and Noller, H. F. |
| | | | nonsense codons. | | (1995) Nature 377, 309-314. 3. |
| | | | | | O'Connor, M., Brunelli, C. A., Firpo, M. A., |
| | | | | | Gregory, S. T., Lieberman, K. R., Lodmell, J. S., |
| | | | | | Moine, H., Van Ryk, D. I., and |
| | | | | | Dahlberg, A. E. (1995) Cell Biol. 73, 859-868. |
| 23S | 2253 | “G to U” | Promoted | E. coli | 1. Lieberman, K. R. and Dahlberg, A. E. |
| | | | frameshifting and | | (1994) J. Biol. Chem. 269, 16163-16169. |
| | | | readthrough of | | 2. Samaha, R. R., Green R., and Noller, H. F. |
| | | | nonsense codons. | | (1995) Nature 377, 309-314. 3. |
| | | | | | O'Connor, M., Brunelli, C. A., Firpo, M. A., |
| | | | | | Gregory, S. T., Lieberman, K. R., Lodmell, J. S., |
| | | | | | Moline, H., Van Ryk, D. I., and |
| | | | | | Dahlberg, A. E. (1995) Cell Biol. 73, 859-868. |
| 23S | 2253 | “G to A” | 19% of control level | E. coli | Porse, B. T., Thi-Ngoc, H. P., and Garrett, R. A. |
| | | | peptidyl transferase | | (1996) J. Mol. Biol. 264: 472-486. |
| | | | activity. | | |
| 23S | 2253 | “G to C” | Severely detrimental | E. coli | 1. Lieberman, K. R. and Dahlberg, A. E. |
| | | | to cell growth; | | (1994) J. Biol. Chem. 269, 16163-16169. |
| | | | reduced rate of | | 2. Samaha, R. R., Green R., and Noller, H. F. |
| | | | peptide bond | | (1995) Nature 377, 309-314. 3. |
| | | | formation in vitro. | | O'Connor, M., Brunelli, C. A., Firpo, M. A., |
| | | | Double mutations | | Gregory, S. T., Lieberman, K. R., Lodmell, J. S., |
| | | | (C2253C/2252C). | | Moine, H., Van Ryk, D. I., and |
| | | | | | Dahlberg, A. E. (1995) Cell Biol. 73, 859-868. |
| 23S | 2253 | “G to U” | Less than 5% control | E. coli | Porse, B. T., Thi-Ngoc, H. P., and Garrett, R. A. |
| | | | level peptidyl | | (1996) J. Mol. Biol. 264: 472-486. |
| | | | transferase activity. | | |
| 23S | 2253 | “G to A” | Induced DMS | E. coli | Gregory ST, Dahlberg AE, 1999. |
| | | | reactivity; enhanced | | Mutations in the Conserved P Loop |
| | | | CMCT reactivity in | | Perturb the conformation of Two |
| | | | G2238; induced | | Structural Elements in the Peptidyl |
| | | | kethoxal and CMCT | | Transferase center of 23 S Ribosomal |
| | | | reactivity in G2269 | | RNA. J. Mol. Biol. 285: 1475-1483. |
| | | | and G2271; induced | | |
| | | | CMCT reactivity in | | |
| | | | U2272; induced | | |
| | | | kethoxal reactivity in | | |
| | | | G2409 and G2410. | | |
| 23S | 2253 | “G to C” | Induced DMS | E. coli | Gregory ST, Dahlberg AE, 1999. |
| | | | reactivity; enhanced | | Mutations in the Conserved P Loop |
| | | | CMCT reactivity in | | Perturb the Conformation of Two |
| | | | G2238; induced | | Structural Elements in the Peptidyl |
| | | | kethoxal and CMCT | | Transferase Center of 23 S Ribosomal |
| | | | reactivity in G2269 | | RNA. J. Mol. Biol. 285: 1475-1483. |
| | | | and G2271; induced | | |
| | | | CMCT reactivity in | | |
| | | | U2272; induced | | |
| | | | kethoxal reactivity in | | |
| | | | G2409 and G2410. | | |
| 23S | 2438 | “U to A” | Amicetin resistance | Halobacterium | Leviev, I. G., Rodriguez-Fonseca, C., |
| | | | and reduced growth | halobium | Phan, H., Garrett, R. A., Heliek, G., Noller, H. F., |
| | | | rate. | | and Mankin, A. S (1994) EMBO J. 13, |
| | | | | | 1682-1686. |
| 23S | 2438 | “U to C” | Amicetin resistance. | Halobacterium | Leviev, I. G., Rodriguez-Fonseca, C., |
| | | | | halobium | Phan, H., Garrett, R. A., Heliek, G., Noller, H. F., |
| | | | | | and Mankin, A. S (1994) EMBO J. 13, |
| | | | | | 1682-1686. |
| 23S | 2438 | “U to G” | Unstable in presence | Halobacterium | Leviev, I. G., Rodriguez-Fonseca, C., |
| | | | or absence of | halobium | Phan, H., Garrett, R. A., Heliek, G., Noller, H. F., |
| | | | amicetin | | and Mankin, A. S (1994) EMBO J. 13, |
| | | | | | 1682-1686. |
| 23S | 2447 | “G to A” | Chloramphenicol | Yeast | Dujon, B. (1980) Cell 20, 185-197. |
| | | | resistance. | mitochondria | |
| 23S | 2447 | “G to C” | Anisomycin | Halobacterium | Hummel, H. and Böck, A. (1987) |
| | | | resistance. | halobium | Biochimie 69, 857-861. |
| 23S | 2450 | “A to C” | Lethal. | E. coli | Vester, B. and Garrett, R. A. (1988) EMBO |
| | | | | | J. 7, 3577-3587. |
| 23S | 2451 | “A to U” | Chloramphenicol | Mouse | Kearsey, S. E. and Craig, I. W. (1981) |
| | | | resistance. | mitochondria | Nature (London) 290: 607-608. |
| 23S | 2451 | “A to G” | Like A2451G | E. coli | Bocchetta M, Xiong L, Mankin AS. 1998. |
| | | | | | 23S rRNA positions essential for tRNA |
| | | | | | binding in ribosomal functional sites. Proc. |
| | | | | | Natl. Acad. Sci. 95: 3525-3530. |
| 23S | 2451 | “A to C” | Like A2451G | E. coli | Bocchetta M, Xiong L, Mankin AS. 1998. |
| | | | | | 23S rRNA positions essential for tRNA |
| | | | | | binding in ribosomal functional sites. Proc. |
| | | | | | Natl. Acad. Sci. 95: 3525-3530. |
| 23S | 2452 | “C to A” | Chloramphenicol | Human | Blanc, H., Wright C. T., Bibb M. J., Wallace D. C., |
| | | | resistance. | mitochondria | and Clayton D. A. (1981) Proc. Natl. |
| | | | | | Acad. Sci. USA 78, 3789-3793. |
| 23S | 2452 | “C to U” | Animosycin resistance. | Halobacterium | Hummel, H. and Böck, A. (1987) Biochimie |
| | | | | | 69, 857-861. |
| 23S | 2452 | “C to U” | Animosycin resistance | Tetrahymena | Sweeney, R., Yao, C. H., and Yao, M. C. |
| | | | | thermophilia | (1991) Genetics 127: 327-334. |
| 23S | 2452 | “C to U” | Chloramphenicol | Halobacterium | Mankin, A. S. and Garrett, R. A. (1991) J. |
| | | | resistance. | halobium | Bacteriol. 173: 3559-3563. |
| 23S | 2452 | “C to U” | Chloramphenicol | Mouse | Slott, E. F., Shade R. O., and Lansman, R. A. |
| | | | resistance | mitochondria | (1983) Mol. Cell. Biol. 3, 1694-1702. |
| 23S | 2452 | “C to U” | Low level sparsomycin | Halobacterium | Tan, G. T., DeBlasio, A., and Mankin, A. S. |
| | | | resistance | halobium | (1996) J. Mol. Biol. 261, 222-230. |
| 23S | 2452 | C to U | Cbm-R, Lin-R | Sulfolobus | Aagaard, C., H. Phan, S. Trevisanato, and |
| | | | | acidocaldarius | R. A. Garrett. 1994. A spontaneous point |
| | | | | | mutation in the single 23S rRNA gene of |
| | | | | | the thermophilic arachaeon Sulfolobus |
| | | | | | acidocaldarius confers multiple drug |
| | | | | | resistance. J. Bacteriol. 176: 7744-7747. |
| 23S | 2453 | “A to C” | Anisomycin resistance | Halobacterium | Hummel, H. and Böck, A. (1987) Biochimie |
| | | | | halobium | 69, 857-861. |
| 23S | 2492 | “U to A” | Frameshift | E. coli | O'Connor, M. and Dahlberg, A. E. (1996) |
| | | | suppressors. | | Nucleic Acids Res. 24, 2701-2705. |
| 23S | 2492 | “U to C” | Frameshift | E. coli | O'Connor, M. and Dahlberg, A. E. (1996) |
| | | | suppressors. | | Nucleic Acids Res. 24, 2701-2705. |
| 23S | 2493 | “del U” | (With A2058G and | E. coli | O'Connor, M. and Dahlberg, A. E. (1996) |
| | | | erythromycin) Lethal | | Nucleic Acids Res. 24, 2701-2705. |
| | | | growth effects. | | |
| | | | Frameshift | | |
| | | | suppressors. | | |
| 23S | 2493 | “U to A” | (With A2058G and | E. coli | 1. Porse, B. T. and Garrett, R. A. (1995) J. |
| | | | erythromycin) Lethal | | Mol. Biol. 249, 1-10. 2. O'Connor, M., |
| | | | growth effects. | | Brunelli, C. A., Firpo, M. A., Gregory, S. T., |
| | | | Frameshift suppressors | | Lieberman, K. R., Lodmell, J. S., Moine, H., |
| | | | | | Van Ryk, D. I., and Dahlberg, A. E. (1995) |
| | | | | | Biochem. Cell Biology 73, 859-868. |
| 23S | 2493 | “U to C” | (With A2058G and | E. coli | 1. Porse, B. T. and Garrett, R. A. (1995) J. |
| | | | erythromycin) Lethal | | Mol. Biol. 249, 1-10. 2. O'Connor, M., |
| | | | growth effects. | | Brunelli, C. A., Firpo, M. A., Gregory, S. T., |
| | | | Frameshift suppressors | | Lieberman, K. R., Lodmell, J. S., Moine, H., |
| | | | | | Van Ryk, D. I., and Dahlberg, A. E. (1995) |
| | | | | | Biochem. Cell Biology 73, 859-868. |
| | | | | | 3.O'Connor, M. and Dahlberg, A. E. (1996) |
| | | | | | Nucleic Acids Res. 24, 2701-2705 |
| 23S | 2493 | “U to C” | (With A2058G and | E. coli | O'Connor, M. and Dahlberg, A. E. (1996) |
| | | | erythromycin) Lethal | | Nucleic Acids Res. 24, 2701-2705 |
| | | | growth effects. | | |
| | | | Frameshift suppressors | | |
| 23S | 2493 | “U to G” | (With A2058G and | E. coli | O'Connor, M. and Dahlberg, A. E. (1996) |
| | | | erythromycin) Lethal | | Nucleic Acids Res. 24, 2701-2705 |
| | | | growth effects. | | |
| | | | Frameshift suppressors | | |
| 23S | 2493 | “U to A” | (With A2058G and | E. coli | O'Connor, M. and Dahlberg, A. E. (1996) |
| | | | erythromycin) Lethal | | Nucleic Acids Res. 24, 2701-2705 |
| | | | growth effects. | | |
| | | | Frameshift suppressors | | |
| 23S | 2493 | “U to C” | Increased misreading. | E. coli | O'Connor, M. and Dahlberg, A. E. (1996) |
| | | | Double mutation | | Nucleic Acids Res. 24, 2701-2705 |
| | | | (U2493C/G2458A) | | |
| 23S | 2493 | “U to C” | Increased misreading. | E. coli | O'Connor, M. and Dahlberg, A. E. (1996) |
| | | | Double mutation | | Nucleic Acids Res. 24, 2701-2705 |
| | | | (U2493C/G2458C) | | |
| 23S | 2497 | “A to G” | (With A2058G and | E. coli | Porse, B. T. and Garrett, R. A. (1995) J. |
| | | | erythromycin) Reduced | | Mol. Biol. 249, 1-10. |
| | | | growth rate. | | |
| 23S | 2499 | “C to U” | Sparsomycin | Halobacterium | Tan, G. T., DeBlasio, A. and Mankin, A. S. |
| | | | resistance | halobium | (1996) J. Mol. Biol. 261, 222-230 |
| 23S | 2500 | U2500A/C2501A | Inhibits binding of 1A | E. coli | Porse BT, Garrett RA. 1999. Sites of |
| | | | streptogramin B, | | Interaction of Streptogramin A and B |
| | | | antibiotic pristinamycin | | Antibiotics in the Peptidyl Transferase |
| | | | 1A on peptidyl | | Loop of 23 S rRNA and the Synergism of |
| | | | transferase loop | | their Inhibitory Mechanisms. J. Mol. Biol. |
| | | | causing inhibition of | | 286: 375-387. |
| | | | peptide elongation. c | | |
| 23S | 2500 | U2500A/C2501G | Like U2500A/C2501A. c | E. coli | Porse BT, Garrett RA. 1999. Sites of |
| | | | | | Interaction of Streptogramin A and B |
| | | | | | Antibiotics in the Peptidyl Transferase |
| | | | | | Loop of 23 S rRNA and the Synergism of |
| | | | | | their Inhibitory Mechanisms. J. Mol. Biol. |
| | | | | | 286: 375-387. |
| 23S | 2500 | U2500A/C2501U | Like U2500A/C2501A. c | E. coli | Porse BT, Garrett RA. 1999. Sites of |
| | | | | | Interaction of Streptogramin A and B |
| | | | | | Antibiotics in the Peptidyl Transferase |
| | | | | | Loop of 23 S rRNA and the Synergism of |
| | | | | | their Inhibitory Mechanisms. J. Mol. Biol. |
| | | | | | 286: 375-387. |
| 23S | 2500 | U2500G/C2501A | Like U2500A/C2501A. c | E. coli | Porse BT, Garrett RA. 1999. Sites of |
| | | | | | Interaction of Streptogramin A and B |
| | | | | | Antibiotics in the Peptidyl Transferase |
| | | | | | Loop of 23 S rRNA and the Synergism of |
| | | | | | their Inhibitory Mechanisms. J. Mol. Biol. |
| | | | | | 286: 375-387. |
| 23S | 2500 | U2500G/C2501G | Like U2500A/C2501A. c | E. coli | Porse BT, Garrett RA. 1999. Sites of |
| | | | | | Interaction of Streptogramin A and B |
| | | | | | Antibiotics in the Peptidyl Transferase |
| | | | | | Loop of 23 S rRNA and the Synergism of |
| | | | | | their Inhibitory Mechanisms. J. Mol. Biol. |
| | | | | | 286: 375-387. |
| 23S | 2500 | U2500G/C2501U | Like U2500A/C2501A. c | E. coli | Porse BT, Garrett RA. 1999. Sites of |
| | | | | | Interaction of Streptogramin A and B |
| | | | | | Antibiotics in the Peptidyl Transferase |
| | | | | | Loop of 23 S rRNA and the Synergism of |
| | | | | | their Inhibitory Mechanisms. J. Mol. Biol. |
| | | | | | 286: 375-387. |
| 23S | 2500 | U2500C/C2501A | Like U2500A/C2501A. c | E. coli | Porse BT, Garrett RA. 1999. Sites of |
| | | | | | Interaction of Streptogramin A and B |
| | | | | | Antibiotics in the Peptidyl Transferase |
| | | | | | Loop of 23 S rRNA and the Synergism of |
| | | | | | their Inhibitory Mechanisms. J. Mol. Biol. |
| | | | | | 286: 375-387. |
| 23S | 2500 | U2500C/C2501G | Like U2500A/C2501A. c. | E. coli | Porse BT, Garrett RA. 1999. Sites of |
| | | | | | Interaction of Streptogramin A and B |
| | | | | | Antibiotics in the Peptidyl Transferase |
| | | | | | Loop of 23 S rRNA and the Synergism of |
| | | | | | their Inhibitory Mechanisms. J. Mol. Biol. |
| | | | | | 286: 375-387. |
| 23S | 2500 | U2500C/C2501A | Like U2500A/C2501A. c | E. coli | Porse BT, Garrett RA. 1999. Sites of |
| | | | | | Interaction of Streptogramin A and B |
| | | | | | Antibiotics in the Peptidyl Transferase |
| | | | | | Loop of 23 S rRNA and the Synergism of |
| | | | | | their Inhibitory Mechanisms. J. Mol. Biol. |
| | | | | | 286: 375-387. |
| 23S | 2502 | “G to A” | Decreased growth rate | E. coli | Vester, B. and Garrett, R. A. (1988) EMBO |
| | | | | | J. 7, 3577-3587 |
| 23S | 2503 | “A to C” | Chloramphenicol | Yeast | Dujon, B. (1980) Cell 20, 185-197 |
| | | | resistance | mitochondria | |
| 23S | 2503 | “A to C” | Decreased growth rate; | E. coli | Porse, B. T. and Garrett, R. A. (1995) J. |
| | | | CAMr | | Mol. Biol. 249, 1-10. |
| 23S | 2503 | “A to G” | (With A2058G and | E. coli | Porse, B. T. and Garrett, R. A. (1995) J. |
| | | | erythromycin) Slow | | Mol. Biol. 249, 1-10. |
| | | | growth rate. CAMr | | |
| 23S | 2504 | “U to A” | Increased readthrough | E. coli | O'Connor, M., Brunelli, C. A., Firpo, M. A., |
| | | | of stop codons and | | Gregory, S. T., Lieberman, K. R., Lodmell, J. S., |
| | | | frameshifting; lethal | | Moine, H., Van Ryk, D. I., and |
| | | | | | Dahlberg, A. E. (1995) Biochem. Cell |
| | | | | | Biology 73, 859-868. |
| 23S | 2504 | “U to C” | Increased readthrough | E. coli | O'Connor, M., Brunelli, C. A., Firpo, M. A., |
| | | | of stop codons and | | Gregory, S. T., Lieberman, K. R., Lodmell, J. S., |
| | | | frameshifting; lethal | | Moine, H., Van Ryk, D. I., and |
| | | | | | Dahlberg, A. E. (1995) Biochem. Cell |
| | | | | | Biology 73, 859-868. |
| 23S | 2504 | “U to C” | Chloramphenicol | Mouse | Blanc, H., Wright, C. T., Bibb, M. J., |
| | | | resistance | mitochondria | Wallace, D. C., and Clayton, D. A. (1981) |
| | | | | | Proc. Natl. Acad. Sci. USA 78, 3789-3793 |
| 23S | 2504 | “U to C” | Chloramphenicol | Human | Kearsey, S. E., and Craig, I. W. (1981) |
| | | | resistance | mitochondria | Nature (London) 290, 607-608 |
| 23S | 2505 | “G to A” | 14% activity of 70S | E. coli | Porse, B. T., Thi-Ngoc, H. P. and Garrett, R. A. |
| | | | ribosomes | | (1996) J. Mol. Biol. 264, 472-486 |
| 23S | 2505 | “G to C” | (With A1067U and | E. coli | 1. Saarma, U. and Remme, J. (1992) |
| | | | thiostrepton) Temperature | | Nucleic Acids Res. 23, 2396-2403. 2. |
| | | | sensitive growth. a | | Saarma, U., Lewicki, B. T. U., Margus, T., |
| | | | Hypersensitivity to CAM; | | Nigul, S., and Remme, J. (1993) “The |
| | | | increased sensitivity of in | | Translational Apparatus: Structure, |
| | | | vitro translation. Slight | | Function, Regulation and Evolution” 163-172. |
| | | | increase in sensitivity to | | |
| | | | lincomycin. b No effect on | | |
| | | | translational accuracy. | | |
| 23S | 2505 | “G to C” | Excluded from 70S | E. coli | Porse, B. T. Thi-Ngoc, H. P. and Garrett, R. A. |
| | | | ribosomes; 17% activity of | | (1996) J. Mol. Biol. 264, 472-486 |
| | | | 70S ribosomes | | |
| 23S | 2505 | “G to U” | <5% activity of 70S | E. coli | Porse, B. T., Thi-Ngoc, H. P. and Garrett, R. A. |
| | | | ribosomes | | (1996) J. Mol. Biol. 264, 472-486 |
| 23S | 2505 | “G to A” | Conferred resistance to | E. coli | Hansen LH, Mauvais P, Douthwaite S. |
| | | | the MLS drugs and | | 1999. The macrolide-kelotide antibiotic |
| | | | chloramphenicol. | | binding site is formed by structures in |
| | | | | | domains II and V of 23S ribosomal RNA. |
| | | | | | Molecular Microbiology 31 (2): 623-631. |
| 23S | 2505 | “G to C” | Like G2505A. | E. coli | Hansen LH, Mauvais P, Douthwaite S. |
| | | | | | 1999. The macrolide-kelotide antibiotic |
| | | | | | binding site is formed by structures in |
| | | | | | domains II and V of 23S ribosomal RNA. |
| | | | | | Molecular Microbiology 31 (2): 623-631. |
| 23S | 2505 | “G to U” | Like G2505A | E. coli | Hansen LH, Mauvais P, Douthwaite S. |
| | | | | | 1999. The macrolide-kelotide antibiotic |
| | | | | | binding site is formed by structures in |
| | | | | | domains II and V of 23S ribosomal RNA. |
| | | | | | Molecular Microbiology 31 (2): 623-631. |
| 23S | 2506 | “U to A” | Dominant lethal; 5% | E. coli | Porse, B. T., Thi-Ngoc, H. P. and Garrett, R. A. |
| | | | activity of 70S ribosomes | | (1996) J. Mol. Biol. 264, 472-486 |
| 23S | 2508 | “A to U” | Eryr, Cdr, Cms; abolishes | E. coli | 1. Sigmund, C. D., Ettayebi, M., and |
| | | | methylation of 23S rRNA | | Morgan, E. A. (1984) Nucleic Acids Res. |
| | | | by ErmE. | | 12, 4653-4663. 2. Vannuffel, P., Di |
| | | | | | Giambattista, M., and Cocito, C. (1992) |
| | | | | | J. Biol. Chem. 267, 16114-16120. 3. |
| | | | | | Douthwaite, S. and Aagaard, C. (1993) J. |
| | | | | | Mol. Biol. 232, 725-731. 4. Vester, B., |
| | | | | | Hansen, L. H., and Douthwaite, S. (1995) |
| | | | | | RNA 1, 501-509. |
| 23S | 2508 | “G to U” | Control level peptidyl | E. coli | 1. Porse, B. T. and Garrett, R. A. (1995) J. |
| | | | transferase activity | | Mol. Biol. 249, 1-10. 2. Porse, B. T., Thi- |
| | | | | | Ngoc, H. P. and Garrett, R. A. (1996) J. |
| | | | | | Mol. Biol. 264, 472-486 |
| 23S | 2514 | “U to C” | Control level peptidyl | E. coli | Porse, B. T. and Garrett, R. A. (1995) J. |
| | | | transferase activity | | Mol. Biol. 249, 1-10. |
| 23S | 2516 | “A to U” | Control level peptidyl | E. coli | Porse, B. T. and Garrett, R. A. (1995) J. |
| | | | transferase activity | | Mol. Biol. 249, 1-10. |
| 23S | 2528 | “U to A” | (With A2058G and | E. coli | Porse, B. T. and Garrett, R. A. (1995) J. |
| | | | erythromycin) Slow growth | | Mol. Biol. 249, 1-10. |
| | | | rate. Control level peptidyl | | |
| | | | transferase activity | | |
| 23S | 2528 | “U to C” | Control level peptidyl | E. coli | Porse, B. T. and Garrett, R. A. (1995) J. |
| | | | transferase activity | | Mol. Biol. 249, 1-10. |
| 23S | 2530 | “A to G” | (With A2058G and | E. coli | Porse, B. T. and Garrett, R. A. (1995) J. |
| | | | erythromycin) Slow growth | | Mol. Biol. 249, 1-10. |
| | | | rate. | | |
| 23S | 2546 | “U to C” | Control level peptidyl | E. coli | Porse, B. T. and Garrett, R. A. (1995) J. |
| | | | transferase activity. | | Mol. Biol. 249, 1-10. |
| 23S | 2550 | “G to A” | (With A2058G and | E. coli | Porse, B. T. and Garrett, R. A. (1995) J. |
| | | | erythromycin) Slow growth | | Mol. Biol. 249, 1-10. |
| | | | rate. | | |
| 23S | 2552 | “U to A” | (With A2058G and | E. coli | Porse, B. T. and Garrett, R. A. (1995) J. |
| | | | erythromycin) Slow growth | | Mol. Biol. 249, 1-10. |
| | | | rate. | | |
| 23S | 2555 | “U to A” | Stimulates readthrough of | E. coli | 1. O'Connor, M. and Dalhberg, A. E. |
| | | | stop codons and | | (1993) Proc. Natl. Acad. Sci. USA 90, |
| | | | frameshifting; U to A is | | 9214-9218 2. O'Connor, M., Brunelli, C. A., |
| | | | trpE91 frameshift | | Firpo, M. A., Gregory, S. T., |
| | | | suppressor; viable in low | | Lieberman, K. R., Lodmell, J. S., Moine, H., |
| | | | copy number plasmids, | | Van Ryk, D. I., and Dahlberg, A. E. |
| | | | but lethal when expressed | | (1995) Biochem. Cell Biology 73, 859-868. |
| | | | constitutively from lambda | | |
| | | | pL promoter | | |
| 23S | 2555 | “U to C” | (With A2058G and | E. coli | Porse, B. T. and Garrett, R. A. (1995) J. |
| | | | erythromycin) Slow growth | | Mol. Biol. 249, 1-10. |
| | | | rate. Control level peptidyl | | |
| | | | transferase activity | | |
| 23S | 2555 | “U to C” | no effect | E. coli | O'Connor, M. and Dalhberg, A. E. (1993) |
| | | | | | Proc. Natl. Acad. Sci. USA 90, 9214-9218 |
| 23S | 2557 | “G to A” | (With A2058G and | E. coli | Porse, B. T. and Garrett, R. A. (1995) J. Mol. |
| | | | erythromycin) Slow growth | | Biol. 249, 1-10. |
| | | | rate. Intermediate decrease | | |
| | | | in peptidyl transferase | | |
| | | | activity. | | |
| 23S | 2565 | “A to U” | (With A2058G and | E. coli | Porse, B. T. and Garrett, R. A. (1995) J. Mol. |
| | | | erythromycin) Slow growth | | Biol. 249, 1-10. |
| | | | rate. Very low peptidyl | | |
| | | | transferase activity. | | |
| 23S | 2580 | “U to C” | (With A2058G and | E. coli | Porse, B. T. and Garrett, R. A. (1995) J. Mol. |
| | | | erythromycin) Lethal growth | | Biol. 249, 1-10. |
| | | | effects. No peptidyl | | |
| | | | transferase activity. | | |
| 23S | 2581 | “G to A” | Dominant lethal inhibition of | E. coli | 1. Spahn, C., Reeme, J., Schafer, M. and |
| | | | puromycin in reaction | | Nierhaus, K. (1996) J. Biol. Chem. 271, |
| | | | | | 32849-32856 2. Spahn, C., Reeme, J., |
| | | | | | Schafer, M. and Nierhaus, K. (1996) J. Biol. |
| | | | | | Chem. 271, 32857-32862 |
| 23S | 2584 | “U to A” | Deleterious; 20% activity of | E. coli | Porse, B. T., Thi-Ngoc, H. P. and Garrett, R. A. |
| | | | 70S ribosomes | | (1996) J. Mol. Biol. 264, 472-486 |
| 23S | 2584 | “U to C” | Deleterious; 20% activity of | E. coli | Porse, B. T., Thi-Ngoc, H. P. and Garrett, R. A. |
| | | | 70S ribosomes | | (1996) J. Mol. Biol. 264, 472-486 |
| 23S | 2584 | “U to G” | (With A2058G and | E. coli | Porse, B. T. and Garrett, R. A. (1995) J. Mol. |
| | | | erythromycin) Lethal growth | | Biol. 249, 1-10. |
| | | | effects. No peptidyl | | |
| | | | transferase activity. | | |
| 23S | 2589 | “A to G” | (With A2058G and | E. coli | Porse, B. T. and Garrett, R. A. (1995) J. |
| | | | erythromycin) Slow growth | | Mol. Biol. 249, 1-10. |
| | | | rate. Strong reduction in | | |
| | | | peptidyl transferase | | |
| | | | activity. | | |
| 23S | 2602 | A2602C/C2501A | Inhibits binding of 1A | E. coli | Porse BT, Kirillov SV, Awayez MJ, |
| | | | streptogramin B, antibiotic | | Garrett RA. 1999. UV-induced |
| | | | pristinamycin 1A on | | modifications in the peptidyl |
| | | | peptidyl transferase loop | | transferase loop of 23S rRNA |
| | | | causing inhibition of | | dependent on binding of the |
| | | | peptide elongation. c | | streptogramin B antibiotic pristinamycin |
| | | | | | IA. RNA 5: 585-595. |
| 23S | 2602 | A2602C/C2501U | Like A2602C/C2501A. c | E. coli | Porse BT, Kirillov SV, Awayez MJ, |
| | | | | | Garrett RA. 1999. UV-induced |
| | | | | | modifications in the peptidyl |
| | | | | | transferase loop of 23S rRNA |
| | | | | | dependent on binding of the |
| | | | | | streptogramin B antibiotic pristinamycin |
| | | | | | IA. RNA 5: 585-595. |
| 23S | 2602 | A2602C/C2501G | Like A2602C/C2501A. c | E. coli | Porse BT, Kirillov SV, Awayez MJ, |
| | | | | | Garrett RA. 1999. UV-induced |
| | | | | | modifications in the peptidyl |
| | | | | | transferase loop of 23S rRNA |
| | | | | | dependent on binding of the |
| | | | | | streptogramin B antibiotic pristinamycin |
| | | | | | IA. RNA 5: 585-595. |
| 23S | 2602 | A2602U/C2501A | Like A2602C/C2501A. c | E. coli | Porse BT, Kirillov SV, Awayez MJ, |
| | | | | | Garrett RA. 1999. UV-induced |
| | | | | | modifications in the peptidyl |
| | | | | | transferase loop of 23S rRNA |
| | | | | | dependent on binding of the |
| | | | | | streptogramin B antibiotic pristinamycin |
| | | | | | IA. RNA 5: 585-595. |
| 23S | 2602 | A2602U/C2501U | Like A2602C/C2501A. c | E. coli | Porse BT, Kirillov SV, Awayez MJ, |
| | | | | | Garrett RA. 1999. UV-induced |
| | | | | | modifications in the peptidyl |
| | | | | | transferase loop of 23S rRNA |
| | | | | | dependent on binding of the |
| | | | | | streptogramin B antibiotic pristinamycin |
| | | | | | IA. RNA 5: 585-595. |
| 23S | 2602 | A2602U/C2501G | Like A2602C/C2501A. c | E. coli | Porse BT, Kirillov SV, Awayez MJ, |
| | | | | | Garrett RA. 1999. UV-induced |
| | | | | | modifications in the peptidyl |
| | | | | | transferase loop of 23S rRNA |
| | | | | | dependent on binding of the |
| | | | | | streptogramin B antibiotic pristinamycin |
| | | | | | IA. RNA 5: 585-595. |
| 23S | 2602 | A2602G/C2501A | Like A2602C/C2501A. c | E. coli | Porse BT, Kirillov SV, Awayez MJ, |
| | | | | | Garrett RA. 1999. UV-induced |
| | | | | | modifications in the peptidyl |
| | | | | | transferase loop of 23S rRNA |
| | | | | | dependent on binding of the |
| | | | | | streptogramin B antibiotic pristinamycin |
| | | | | | IA. RNA 5: 585-595. |
| 23S | 2602 | A2602G/C2501U | Like A2602C/C2501A. c | E. coli | Porse BT, Kirillov SV, Awayez MJ, |
| | | | | | Garrett RA. 1999. UV-induced |
| | | | | | modifications in the peptidyl |
| | | | | | transferase loop of 23S rRNA |
| | | | | | dependent on binding of the |
| | | | | | streptogramin B antibiotic pristinamycin |
| | | | | | IA. RNA 5: 585-595. |
| 23S | 2602 | A2602G/C2501G | Like A2602C/C2501A. c | E. coli | Porse BT, Kirillov SV, Awayez MJ, |
| | | | | | Garrett RA. 1999. UV-induced |
| | | | | | modifications in the peptidyl |
| | | | | | transferase loop of 23S rRNA |
| | | | | | dependent on binding of the |
| | | | | | streptogramin B antibiotic pristinamycin |
| | | | | | IA. RNA 5: 585-595. |
| 23S | 2611 | “C to G” | Erythromycin and | Chlamydomonas | Gauthier, A., Turmel, M. and Lemieux, C. |
| | | | spiramycin resistance | reinhardtii | (1988) Mol. Gen. Genet. 214, 192-197. |
| 23S | 2611 | “C to G” | Erythromycin and | Yeast | Sor, F. and Fukahara, H. (1984) |
| | | | spiramycin resistance | mitochondria | Nucleic Acids Res. 12, 8313-8318. |
| 23S | 2611 | “C to U” | Eryr and low level | Chlamydomonas | Harris, E. H., Burkhart, B. D., Gillham, N. W. |
| | | | lincomycin and clindamycin | reinhardtii | and Boynton, J. E. (1989) |
| | | | resistance | | Genetics 123, 281-292 |
| 23S | 2611 | “C to G” | Eryr and low level | Chlamydomonas | Harris, E. H., Burkhart, B. D., Gillham, N. W. |
| | | | lincomycin and clindamycin | reinhardtii | and Boynton, J. E. (1989) |
| | | | resistance | | Genetics 123, 281-292 |
| 23S | 2611 | “C to U” | Slightly Eryr; reduced | E. coli | Vester, B., Hansen, L. H., and |
| | | | methylation Double | | Douthwaite, S. (1995) RNA 1, 501-509 |
| | | | mutation (C2611U/ | | |
| | | | G2057A) | | |
| 23S | 2611 | C to G | Ery-R, Spi-LR | Chlamydomonas | Gauthier, A., M. Turmel, and C. Lemieux. |
| | | | | moewusii | 1988. Mapping of chloroplast |
| | | | | chl. | mutations conferring resistance to |
| | | | | | antibiotics in Chlamydomonas: |
| | | | | | evidence for a novel site of |
| | | | | | streptomycin resistance in the small |
| | | | | | subunit rRNA. Mol. Gen. Genet. |
| | | | | | 214: 192-197. |
| 23S | 2611 | C to G/U | Ery-R, Lin-MR | Chlamydomonas | Harris, E. H., B. D. Burkhart, N. W. Gillham, |
| | | | | reinhardtii | and J. E. Boynton. 1989. |
| | | | | chl. | Antibiotic resistance mutations in the |
| | | | | | chloroplast 16S and 23S rRNA genes |
| | | | | | of Chlamydomonas reinhardtii: |
| | | | | | correlation of genetic and physical |
| | | | | | maps of the chloroplast genome. |
| | | | | | Genetics. |
| 23S | 2611 | C to U | Ery-R, Spi-S, Tyl-S, Lin-S | Escherichia | Vannuffel, P., M. Di Giambattista, E. A. Morgan, |
| | | | | coli | and C. Cocito. 1992. |
| | | | | | Identification of a single base change |
| | | | | | in ribosomal RNA leading to |
| | | | | | erythromycin resistance. J. Biol. Chem. |
| | | | | | 267: 8377-8382. |
| 23S | 2611 | C to A/G | Mac-R, SB-S | Streptococcus | Tait-Kamradt, A., T. Davies, M. Cronan, |
| | | | | pneumoniae | M. R. Jacobs, P. C. Appelbaum, and J. Sutcliffe. |
| | | | | | 2000. Mutations in 23S rRNA |
| | | | | | and L4 ribosomal protein account for |
| | | | | | resistance in Pneumococcal strains |
| | | | | | selected in vitro by macrolide passage. |
| | | | | | Antimicrobial Agents and |
| 23S | 2611 | C to G | Ery-R, Spi-R | Saccharomyces | Sor, F., and H. Fukuhara. 1984. |
| | | | | cerevisiae | Erythromycin and spiramycin |
| | | | | mit. | resistance mutations of yeast |
| | | | | | mitochondria: nature of the rib2 locus |
| | | | | | in the large ribosomal RNA gene. |
| | | | | | Nucleic Acids Res. 12: 8313-8318. |
| 23S | 2611 | C to U | Ery-S, Spi-R | Saccharomyces | Sor, F., and H. Fukuhara. 1984. |
| | | | | cerevisiae | Erythromycin and spiramycin |
| | | | | mit. | resistance mutations of yeast |
| | | | | | mitochondria: nature of the rib2 locus |
| | | | | | in the large ribosomal RNA gene. |
| | | | | | Nucleic Acids Res. 12: 8313-8318. |
| 23S | 2661 | “G to C” | Decreased misreading; | E. coli | 1. Tapprich, W. E. and Dalhberg, A. E. |
| | | | streptomycin dependent | | (1990) EMBO J. 9, 2649-2655 2. Tapio, S. |
| | | | when expressed with Smr, | | and Isaksson, L. A. (1991) Eur. J. |
| | | | hyperaccurate S12 | | Biochem. 202, 981-984 3. Melancon, P., |
| | | | mutation. | | Tapprich, W. and Brakier-Gingras, L. |
| | | | | | (1992) J. Bacteriol. 174, 7896-7901 4. |
| | | | | | Bilgin, N. and Ehrenberg, M. (1994) J. |
| | | | | | Mol. Biol. 235, 813-824 5. O'Connor, M., |
| | | | | | Brunelli, C. A., Firpo, M. A., Gregory, S. T., |
| | | | | | Lieberman, K. R., Lodmell, J. S., |
| | | | | | Moine, H., Van Ryk, D. I., and Dahlberg, A. E. |
| | | | | | (1995) Biochem. Cell Biology 73, |
| | | | | | 859-868. |
| 23S | 2661 | “C to A” | Like C2661 | E. coli | Munishkin A, Wool IG. 1997. The |
| | | | | | ribosome-in-pieces: Binding of |
| | | | | | elongation factor EF-G to |
| | | | | | oligoribonucleotides that mimic the |
| | | | | | sarcin/ricin and thiostrepton domains of |
| | | | | | 23S ribosomal RNA. Proc. Natl. Acad. |
| | | | | | Sci. 94: 12280-12284. |
| 23S | 2661 | “C to G” | Like C2661 | E. coli | Munishkin A, Wool IG. 1997. The |
| | | | | | ribosome-in-pieces: Binding of |
| | | | | | elongation factor EF-G to |
| | | | | | oligoribonucleotides that mimic the |
| | | | | | sarcin/ricin and thiostrepton domains of |
| | | | | | 23S ribosomal RNA. Proc. Natl. Acad. |
| | | | | | Sci. 94: 12280-12284. |
| 23S | 2661 | “C to U” | Like C2661 | E. coli | Munishkin A, Wool IG. 1997. The |
| | | | | | ribosome-in-pieces: Binding of |
| | | | | | elongation factor EF-G to |
| | | | | | oligoribonucleotides that mimic the |
| | | | | | sarcin/ricin and thiostrepton domains of |
| | | | | | 23S ribosomal RNA. Proc. Natl. Acad. |
| | | | | | Sci. 94: 12280-12284. |
| 23S | 2666 | “C to G” | Increased stop codon | E. coli | O'Connor, M. and Dalhberg, A. E. (1996) |
| | | | readthrough and | | Nucleic Acids Res. 24, 2701-2705 |
| | | | frameshifting. a Double | | |
| | | | mutation | | |
| | | | (C2666G/A2654C) | | |
| 23S | 2666 | “C to G” | Minor increase in stop | E. coli | O'Connor, M. and Dalhberg, A. E. (1996) |
| | | | codon readthrough and | | Nucleic Acids Res. 24, 2701-2705 |
| | | | frameshifting. a Double | | |
| | | | mutation | | |
| | | | (C2666G/A2654U) | | |
| 23S | 2666 | “C to U” | Minor increase in stop | E. coli | O'Connor, M. and Dalhberg, A. E. (1996) |
| | | | codon readthrough and | | Nucleic Acids Res. 24, 2701-2705 |
| | | | frameshifting. a Double | | |
| | | | mutation (C2666U/A2654C) | | |
| 23S | 2666 | “C to U” | Significant increase in stop | E. coli | O'Connor, M. and Dalhberg, A. E. (1996) |
| | | | codon readthrough and | | Nucleic Acids Res. 24, 2701-2705 |
| | | | frameshifting. a Double | | |
| | | | mutation | | |
| | | | (C2666U/A2654G) | | |
| 23S | 2666 | “C to U” | Minor increase in stop | E. coli | O'Connor, M. and Dalhberg, A. E. (1996) |
| | | | codon readthrough and | | Nucleic Acids Res. 24, 2701-2705 |
| | | | frameshifting. a Double | | |
| | | | mutation (C2666U/A2654U) | | |

TABLE 3

| | | | | | Effect of resistance on | % Change in |
|---|---|---|---|---|---|---|
| Resistance | | Pathogen | Recomended Etest | Mechanism | fluorescence | Fluorescenc |
| MRSA | Methicillin | Staphylococcus | | Reduced affinity of PBP2 towards penicillins; | Reduction of Penicillin binding | > to ≈80% |
| | Resistant SA | aureus | | nosocomial, Multi-drug-resistant (clindamycin, | | reduction |
| | | | | gentamicin, FQ); Contain SCCmec type I, II, or III; | | |
| | | | | Usually PVL-negative; Virulent (esp. skin and lung) | | |
| CA-MRSA | Community | Staphylococcus | | Multi-drug-resistant (clindamycin, gentamicin, FQ); | Reduction of Penecillin | > to ≈80% |
| | acquired MRSA; | aureus | | Usually only resistant to pen, ox ± eryth ± FQs; Usually | binding. Maintaims binding | reduction of |
| | | | | produce PVL, especially in the US; In general the | capacity for clindamycin and | penicillin, |
| | | | | organisms remain susceptible to clindamycin and to | trimethoprim | clindamycin |
| | | | | trimethoprim sulfa. That's different from a nosocomial | | trimethoprim |
| | | | | pathogen which is usually resistant to one of these antibiotics. | | binding |
| PVL- | Panton-Valentine | Staphylococcus | | Highly abundant toxin, scausing septic shock; large complications | — | — |
| MRSA | leukocidin- | aureus | | | | |
| | MRSA | | | | | |
| BORSA | Border line | Staphylococcus | Oxacillin | | Reduction of Penecillin | > to ≈80% |
| | oxacillin resistant | aureus | | | binding. | reduction of |
| | SA | | | | | penicillin |
| ORSA | Oxacillin | Staphylococcus | Oxacillin | | Reduction of Penecillin | > to ≈80% |
| | resistant SA | aureus | | | binding. | reduction of |
| | | | | | | penicillin |
| VRSA | Vancomycin | Staphylococcus | Vancomycin; | Modified phenotypic features, however, include slower | Increased binding of | |
| | resistant SA | aureus | Teicoplanin | growth rates, a thickened cell wall, and increased levels | Vancomycin | |
| VRS | Vancomycin | Staphylococci | Vancomycin | of PBP2 and PBP2′ (although the degree of cross-linking | Increased binding of | >50% |
| | resistant Staph | (CNS) | | within the thick cell wall seems to be reduced) [58]. | Vancomycin >50% | |
| | | | | Vancomycin resistant strains also seem to have a greater | | |
| | | | | ability to absorb the antibiotic from the outside medium, | | |
| | | | | which may be a consequence of the greater availability | | |
| | | | | of stem peptides in the thick cell wall. In addition, the | | |
| | | | | increased amounts of two PBPs may compete with the | | |
| | | | | antibiotic for the stem peptide substrates, thus | | |
| | | | | aggravating the resistance profile | | |
| VISA | Vancomycin | Staphylococcus | Vancomycin | | Increased binding of | 20-50% |
| | intermediary SA | aureus | | | Vancomycin 20-50% | |
| hVISA | hetero-(resistant) | Staphylococcus | Vancomycin | very rare, but can be susceptible to methicillin and | Binding of penicillin and | |
| | Vancomycin | aureus | | resistant to vancomycin | vancomycin | |
| VRE | Vancomycin | Enterococci | Vancomycin; | Reduced affinity to Van by 3 orders of magnitude | Reduction of vancomycin | >80% |
| | resistant | | Teicoplanin | | binding | reduction |
| ESBL | Extended | Enterobacteriaceae | | Overproduction of β-Lactamases, inhibited by clavulanic | Increased binding of | >80% |
| | Spectrum β- | | | acid | clavulanic acid | increase |
| | Lactamase | | | | | |
| ESBL | Extended | Pseudomonas | Ceftazidime | Overproduction of β-Lactamases, inhibited by clavulanic | Increased binding of | >80% |
| | Spectrum β- | spp. | | acid | clavulanic acid | increase |
| | Lactamase | | | | | |
| ESBL | Extended | Acinetobacter | Ceftazidime | Overproduction of β-Lactamases, inhibited by clavulanic | Increased binding of | >80% |
| | Spectrum β- | | | acid | clavulanic acid | increase |
| | Lactamase | | | | | |
| ESBL | Extended | BCC | Ceftazidime | Overproduction of β-Lactamases, inhibited by clavulanic | Increased binding of | >80% |
| | Spectrum β- | | | acid | clavulanic acid | increase |
| ESBL | Extended | Stenotrophomonas | Ceftazidime | Overproduction of β-Lactamases, inhibited by clavulanic | Increased binding of | >80% |
| | Spectrum β- | maltophilla | | acid | clavulanic acid | increase |
| | Lactamase | | | | | |
| MBL | Metalo-β- | | Imipenem | | — | — |
| | Lactamase | | | | | |
| MLS | macrolide | | | One mechanism is called MLS, macrolide lincosamide | Reduced binding of | >50% |
| | lincosamide | | | streptogramin. And in this situation there is an alteration | macrolides and Ketolides | |
| | streptogramin | | | in a target-binding site at the 23-ribosomal RNA level. | | |
| | | | | resulting in a point mutation or methylation of 23SRNA | | |
| | | | | resulting in reduced binding of macrolides (also | | |
| | | | | Ketolides); organisms with an efflux mechanis will bind | | |
| | | | | macrolides under FISH conditions. They are also | | |
| | | | | sensitive to clindamycin | | |
| DRSP | drug-resistant | Modify | | DR is reported for beta-lactams, macrolides, | Reduction of macrolides | >50% |
| | S. pneumoniae | clavulanic acid, | | chloramphenicol, and sulfonamides | | |
| | | & single gene | | | | |
| | | detection for | | | | |
| | | efflux pump | | | | |
| HLAR | High level | Enterococci | Gentamycin; | | Reduction in Streptomycin binding | >80% reduction |
| | Aminoglycoside | | Streptomycin | | | |
| | Resistance in | | | | | |
| | Enterococci | | | | | |

1. A method for detecting an antibiotic resistance in a predetermined micro-organism in a biological sample, comprising:
(a) contacting the biological sample with a first nucleic acid which is labeled with a first label and which is configured to selectively hybridize with a nucleic acid in the micro-organism under conditions wherein the first nucleic acid and the nucleic acid in the micro-organism selectively hybridize with each other,
(b) identifying the micro-organism by detecting presence of the first label in an individual cell of the micro-organism,
(c) contacting the biological sample with at least one probe or at least one substrate for detection of an antibiotic resistance in a micro-organism, wherein said at least one probe is labeled with a second label, a third label or a fourth label, and wherein said at least one substrate can be modified by a resistance factor, and
(d) determining the antibiotic resistance of the micro-organism by the detection of the presence of one or more of the following labels: the second label, the third label and the fourth label, or/and the modified substrate in an individual cell of the micro-organism,
wherein (b) and (d) are performed simultaneously.

2. The method of claim 1, wherein (a) and (c) are performed simultaneously or separately.

3. The method of claim 1, wherein (a), (b), (c) and (d) are performed simultaneously.

4. The method of claim 1, wherein said at least one probe is selected from
(i) an antibody or/and a fragment thereof being labeled with the second label and capable of selectively binding to a resistance factor, and
wherein the sample is contacted with the antibody or/and the fragment thereof under conditions wherein the antibody or/and the fragment thereof selectively binds to the resistance factor,
(ii) second nucleic acids labeled with the third label and capable of hybridizing with an RNA coding for a resistance factor, and wherein the sample is contacted with the second nucleic acid under conditions wherein the second nucleic acid selectively hybridizes with the RNA, and
(iii) knottins, cystine-knot proteins or/and aptamers labeled with the fourth label and capable of selectively binding to a resistance factor, and wherein the sample is contacted with the knottin, cystine-knot protein or/and aptamer under conditions wherein the knottin, cystine-knot protein or/and aptamer selectively binds to the resistance factor.

5. The method of claim 1, wherein said at least one substrate can be modified by beta-lactamase, wherein the substrate is nitrocefin, and wherein the sample is contacted with the substrate under conditions wherein the resistance factor modifies the substrate.

6. The method of claim 1, wherein the antibiotic resistance is induced in the micro-organism.

7. The method of claim 4, wherein the antibody includes a primary antibody capable of selectively binding to the resistance factor, and a secondary antibody labeled with the second label, wherein the secondary antibody is capable of selectively binding to the primary antibody.

8. The method of claim 4, wherein the antibody or/and the fragment thereof and the second label, or the knottin, cystine-knot protein or/and aptamer and the fourth label, are coupled to a bead.

9. The method of claim 8, wherein in (c) an aggregate is formed, said aggregate comprising more than one micro-organism cell and at least one bead.

10. The method of claim 8, wherein in (c), a plurality of beads is coupled to the micro-organism cell.

11. The method according to claim 4, wherein the antibiotic is selected from the group consisting of aminoglycosides, carbacephems, carbapenems, cephalosporins, glycopeptides, macrolides, monobactams, penicillines, beta-lactam antibiotics, quinolones, bacitracin, sulfonamides, tetracyclines, streptogramines, chloramphenicol, clindamycin, and lincosamide.

12. The method according to claim 1,
wherein the resistance against a beta-lactam antibiotic is detected by an antibody or/and a fragment thereof specifically binding to beta-lactamase or/and PBP2a binding protein, by a nucleic acid specifically hybridizing with an RNA encoding beta-lactamase or/and PBP2a binding protein, or by a knottin, a cystine knot protein or/and an aptamer specifically binding to beta-lactamase or/and PBP2a binding protein.

13. The method according to claim 1, wherein the micro-organism is a Methicillin Resistant Staphylococcus aureus (MRSA) or/and an Oxacillin Resistant Staphylococcus aureus (ORSA), and wherein the antibiotic resistance is detected by the expression of an altered Penicillin Binding Protein 2 or mecA protein.

14. The method according to claim 1,
wherein the micro-organism is selected from Vancomycin Resistant Staphylococcus aureus (VRSA), Vancomycin Resistant Staphylococcus (VRS), Vancomycin Resistant Enterococci (VRE) and Vancomycin Resistant Clostridium difficile (VRCD), and wherein the antibiotic resistance is detected by expression of a peptide selected from vanA protein, vanB protein, vanC protein or/and modified peptidoglycans comprising a D-alanine-D-lactate C-terminus.

15. A kit suitable for detecting an antibiotic resistance in a predetermined micro-organism, comprising
(a) a first nucleic acid capable of selectively hybridizing with a nucleic acid in the micro-organism, wherein the first nucleic acid is labeled with a first label, and
(b) at least one probe or substrate for detection of an antibiotic resistance, said probe or substrate being selected from
(i) an antibody or/and a fragment thereof, and wherein the antibody or/and the fragment thereof is/are labeled with a second label and capable of selectively binding to a resistance factor,
(ii) second nucleic acids labeled with a third label and capable of Preliminary Amendment hybridizing with an RNA coding for a resistance factor,
(iii) knottins, cystine-knot proteins or/and aptamers labeled with a fourth label and capable of selectively binding to a resistance factor, and
(iv) a substrate which can be modified by beta-lactamase, wherein the substrate is nitrocefin.

16. The method of claim 9, wherein in (c), a plurality of beads are coupled to the micro-organism cell.

17. The method of claim 3, wherein said at least one probe is selected from
(i) an antibody or/and a fragment thereof being labeled with the second label and capable of selectively binding to a resistance factor, and
wherein the sample is contacted with the antibody or/and the fragment thereof under conditions wherein the antibody or/and the fragment thereof selectively binds to the resistance factor, (ii) second nucleic acids labeled with the third label and capable of hybridizing
with an RNA coding for a resistance factor, and wherein the sample is contacted with the second nucleic acid under conditions wherein the second nucleic acid selectively hybridizes with the RNA, and
(iii) knottins, cystine-knot proteins or/and aptamers labeled with the fourth label and capable of selectively binding to a resistance factor, and wherein the sample is contacted with the knottin, cystine-knot protein or/and aptamer under conditions wherein the knottin, cystine-knot protein or/and aptamer selectively binds to the resistance factor.
